(19) United States
(12) Patent Application Publication
(10) Pub. No.: US 2026/0347442 A1
(43) Pub. Date: Nov. 5, 2026

(54) DOSAGE AND ADMINISTRATION OF FUSION POLYPEPTIDES FOR TREATMENT OF SICKLE CELL DISEASE

(71) Applicant: Alexion Pharmaceuticals, Inc., Boston, MA (US)

(72) Inventors: Yang DAI, Boston, MA (US); Nader NAJAFIAN, Boston, MA (US); Christoph GASTEYGER, Boston, MA (US); Tong SHEN, Boston, MA (US)

(21) Appl. No.: 19/107,278

(22) PCT Filed: Aug. 31, 2023
(86) PCT No.: PCT/US2023/031615

§ 371 Date: Feb. 27, 2025

Related U.S. Application Data
(60) Provisional application No. 63/374,154, filed on Aug. 31, 2022.
(60) Provisional application No. 63/529,928, filed on Jul. 31, 2023.

(51) Int. Cl. C07K 16/18; A61K 31/17; A61K 39/00; A61P 7/06

(52) U.S. Cl. CPC C07K 16/18; A61K 31/17; A61P 7/06; A61K 2039/505; A61K 2039/54; A61K 2039/545; A61K 2039/55; C07K 2317/31; C07K 2317/569

(57) ABSTRACT

The disclosure provides methods for treating a human patient with sickle cell disease. These methods include administering a properdin binding antibody or antigen-binding fragment thereof to the patient.

### SEQUENCE LISTING

The instant application contains a Sequence Listing which has been submitted electronically in XML format and is hereby incorporated by reference in its entirety. Said XML copy, created on Aug. 25, 2023, is named 51196-032WO3_Sequence_Listing_8_25_23 and is 50,329 bytes in size.

### BACKGROUND

Sickle cell disease (SCD) is the most common monogenic disease worldwide. In some forms, the disease is caused by mutations in the β globin gene, e.g., a single nucleotide mutation in β globin gene resulting in glutamic acid substitution by valine at position 6, the gene that is also responsible for causing beta thalassemia (BT). Despite extensive recognition of the underlying cause of the disease, few treatment options are available to control SCD symptoms. Two main manifestations of SCD, anemia and vaso-occlusion crisis (VOC), affects the mortality, morbidity and quality of life for SCD patients. Although there are two approved treatment options, hydroxyurea and L-glutamine, for SCD patients, they are generally considered suboptimal attenuating disease symptoms. Accordingly, there is a need in the art for new treatments.

### SUMMARY

In a first aspect, the disclosure provides a method for treating a human patient with sickle cell disease including administering a properdin binding antibody or antigen-binding fragment thereof to the patient, wherein the properdin binding antibody or an antigen binding fragment thereof includes CDR1, CDR2, and CDR3 sequences as set forth in SEQ ID NOs: 2, 3, and 4, respectively. In some embodiments, the antibody or antigen binding fragment thereof further includes a human serum albumin binding sequence. In some embodiments, the human serum albumin binding sequence is fused to the C-terminus of the properdin binding antibody or antigen-binding fragment thereof. In some embodiments, the human serum albumin binding sequence is fused to the C-terminus of the properdin binding antibody or antigen-binding fragment thereof by linker. In some embodiments, the linker includes the amino acid sequence of SEQ ID NO: 10. In some embodiments, the human serum albumin binding sequence includes CDR1, CDR2, and CDR3 sequences as set forth in SEQ ID NOs: 5, 6, and 7. In some embodiments, the antibody or antigen binding fragment thereof includes the sequence of SEQ ID NO: 1, or a modification thereof. In some embodiments, the modification comprises conversion of the N-terminal glutamine of the sequence of SEQ ID NO: 1 to pyro-glutamate.

In some embodiments, the antibody or the antigen binding fragment thereof is administered to the patient at a dose of 300 mg. In some embodiments, the antibody or the antigen binding fragment thereof administered to the patient weekly. In some embodiments, the antibody or the antigen binding fragment thereof is administered to the patient for up to 13 weeks (e.g., for 12 weeks). In some embodiments, the antibody or the antigen binding fragment thereof once is administered to the patient every 2 weeks. In some embodiments, the antibody or the antigen binding fragment thereof is administered up to 4 times.

In some embodiments, the antibody or the antigen binding fragment thereof is administered to the patient at a dose of 600 mg. In some embodiments, the antibody or the antigen binding fragment thereof is administered to the patient every 4 weeks. In some embodiments, the antibody or the antigen binding fragment thereof is administered the patient up to 4 times.

In some embodiments, the patient has been clinically diagnosed with sickle cell disease. In some embodiments, the sickle cell disease is HbSS or HbSβ⁰-thalassemia. In some embodiments, the patient is further administered hydroxyurea. In some embodiments, the patient has been receiving a stable dose of hydroxyurea for at least 3 months prior to administration of the antibody or the antigen binding fragment thereof. In some embodiments, the patient has not been administered hydroxyurea for at least 30 days prior to administration the antibody or the antigen binding fragment thereof.

In some embodiments, the patient experiences no treatment emergent adverse events after 12 weeks of treatment. In some embodiments, the patient experiences no serious adverse events after 12 weeks of treatment. In some embodiments, the patient experiences no adverse events after 12 weeks of treatment.

In some embodiments, the method includes further measuring a change in serum concentration of the antibody or antigen binding fragment thereof for up to 30 weeks after starting treatment. In some embodiments, the method further includes measuring a change in blood concentration of an anti-drug antibody for up to 30 weeks after starting treatment. In some embodiments, the patient experiences a change in serum concentration from baseline of total and free properdin after up to 30 weeks of treatment.

In some embodiments, the patient experiences a change in serum concentration from baseline of complement component Ba (Ba), complement component C3a (C3a), or soluble complement component C5B-9 (sC5B9) after 12 weeks of treatment. In some embodiments, the patient experiences a change from baseline in blood or serum concentration of hemopexin, nitric oxide, an inflammatory marker, or a cell adhesion marker after 12 weeks of treatment. In some embodiments, the inflammatory marker includes interleukin-1. In some embodiments, the cell adhesion marker includes soluble P-selectin.

In some embodiments, the patient experiences a change from baseline in hemoglobin levels after 12 weeks of treatment. In some embodiments, the patient experiences a change in serum LDH levels, indirect bilirubin, haptoglobin, or hemopexin after 12 weeks from baseline. In some embodiments, the patient experiences a change in reticulocyte levels after 12 weeks from baseline.

In some embodiments, the patient experiences a reduced rate of vaso-occlusive crisis after 12 weeks of treatment in comparison to baseline. In some embodiments, the patient experiences an increased time to first vaso-occlusive crisis after 12 weeks of treatment in comparison to baseline.

In some embodiments, the antibody or antigen binding fragment thereof is formulated for subcutaneous administration.

In some embodiments, the antibody or antigen binding fragment thereof is formulated at a pH of 5.4 at a concentration of 150 mg/mL in an aqueous solution including 20 nM sodium acetate, 250 mM sucrose, and 0.05% polysorbate-80. In some embodiments, the human patient is between 18 and 65 years of age. In some embodiments, the human patient has a body weight of ≥40 kg.

In another aspect, the disclosure provides an antibody or antigen-binding fragment for use in treating a human patient with sickle cell disease, wherein the use comprises administering a properdin binding antibody or antigen-binding fragment thereof to the patient, wherein the properdin binding antibody or an antigen binding fragment thereof comprises CDR1, CDR2, and CDR3 sequences as set forth in SEQ ID NOs: 2, 3, and 4, respectively. In some embodiments, the antibody or antigen binding fragment thereof for use in treating a human patient with sickle cell disease further comprises a human serum albumin binding sequence. In some embodiments, the human serum albumin binding sequence is fused to the C-terminus of the properdin binding antibody or antigen-binding fragment thereof. In some embodiments, the human serum albumin binding sequence is fused to the C-terminus of the properdin binding antibody or antigen-binding fragment thereof by linker. In some embodiments, the linker comprises the amino acid sequence of SEQ ID NO: 10. In some embodiments, in the human serum albumin binding sequence comprises CDR1, CDR2, and CDR3 sequences as set forth in SEQ ID NOs: 5, 6, and 7. In some embodiments, the antibody or antigen binding fragment thereof for use in treating a human patient with sickle cell disease comprises the sequence of SEQ ID NO: 1, or a modification thereof. In some embodiments, the modification comprises conversion of the N-terminal glutamine of the sequence of SEQ ID NO: 1 to pyro-glutamate.

In some embodiments, the antibody or the antigen binding fragment thereof for use in treating a human patient with sickle cell disease is to be administered to the patient at a dose of 300 mg. In some embodiments, the antibody or the antigen binding fragment thereof for use in treating a human patient with sickle cell disease is to be administered to the patient weekly. In some embodiments, the antibody or the antigen binding fragment thereof for use in treating a human patient with sickle cell disease is to be administered to the patient for up to 13 weeks. In some embodiments, the antibody or the antigen binding fragment thereof for use in treating a human patient with sickle cell disease once is to be administered to the patient every two weeks. In some embodiments, the antibody or the antigen binding fragment thereof for use in treating a human patient with sickle cell disease is to be administered up to four times. In some embodiments, the antibody or the antigen binding fragment thereof is to be administered to the patient at a dose of 600 mg. In some embodiments, the antibody or the antigen binding fragment thereof for use in treating a human patient with sickle cell disease is to be administered to the patient every 4 weeks. In some embodiments, the antibody or the antigen binding fragment thereof for use in treating a human patient with sickle cell disease is to be administered the patient up to 4 times. In some embodiments, the patient has been clinically diagnosed with sickle cells disease. In some embodiments, the sickle cell disease is HbSS or HbSβ⁰-thalassemia. In some embodiments, the patient is further to be administered hydroxyurea. In some embodiments, the patient has been receiving a stable dose of hydroxyurea for at least 3 months prior to administration the antibody or the antigen binding fragment thereof. In some embodiments, the patient has not been administered hydroxyurea for at least 30 days prior to administration the antibody or the antigen binding fragment thereof. In some embodiments, the patient experiences no treatment emergent adverse events after 12 weeks of treatment. In some embodiments, the patient experiences no serious adverse events after 12 weeks of treatment. In some embodiments, the patient experiences no adverse events after 12 weeks of treatment. In some embodiments, the method further comprises measuring a change in serum concentration of the antibody or antigen binding fragment thereof for up to 30 weeks after starting treatment. In some embodiments, the method further comprises measuring a change in blood concentration of an anti-drug antibody for up to 30 weeks after starting treatment. In some embodiments, the patient experiences a change in serum concentration from baseline of total and free properdin after up to 30 weeks of treatment. In some embodiments, the patient experiences a change in serum concentration from baseline of complement component Ba (Ba), complement component C3a (C3a), or soluble complement component C5B-9 (sC5B9) after 12 weeks of treatment. In some embodiments, the patient experiences a change from baseline in blood or serum concentration of hemopexin, nitric oxide, an inflammatory marker, or a cell adhesion marker after 12 weeks of treatment. In some embodiments, the inflammatory marker comprises interleukin-1. In some embodiments, the cell adhesion marker comprises soluble P-selectin. In some embodiments, the patient experiences a change from baseline in hemoglobin levels after 12 weeks of treatment. In some embodiments, the patient experiences a change in serum LDH levels, indirect bilirubin, haptoglobin, or hemopexin after 12 weeks from baseline. In some embodiments, the patient experiences a change in reticulocyte levels after 12 weeks from baseline. In some embodiments, the patient experiences a reduced rate of vaso-occlusive crisis after 12 weeks of treatment in comparison to baseline. In some embodiments, the patient experiences an increased time to first vaso-occlusive crisis after 12 weeks of treatment in comparison to baseline. In some embodiments, the antibody or antigen binding fragment thereof is formulated for subcutaneous administration. In some embodiments, the antibody or antigen binding fragment thereof is formulated at a pH of 5.4 at a concentration of 150 mg/mL in an aqueous solution comprising 20 nM sodium acetate, 250 mM sucrose, and 0.05% polysorbate-80. In some embodiments, the human patient is between 18 and 65 years of age. In some embodiments, the human patient has a body weight of ≥40 kg.

Each and every embodiment can be combined unless the context clearly suggests otherwise. Each and every embodiment can be applied to each and every aspect of the invention unless the context clearly suggests otherwise.

Specific embodiments of the present invention will become evident from the following more detailed description of certain preferred embodiments and the claims.

### BRIEF DESCRIPTION OF THE DRAWINGS

FIG. 1 is a schematic view of the study presented in Example 1.

FIG. 2 is a schematic view of the study presented in Example 2.

FIG. 3 is a graph showing the mean serum complement activity pathway for each cohort described in Example 2 overtime.

### DEFINITIONS

To facilitate the understanding of this disclosure, a number of terms are defined below. Terms defined herein have meanings as commonly understood by a person of ordinary skill in the areas relevant to the disclosure. Terms such as “a,” “an” and “the” are not intended to refer to only a singular entity, but include the general class of which a specific example may be used for illustration. The terminology herein is used to describe specific embodiments, but their usage does not limit the disclosure, except as outlined in the claims.

As used herein, the term “about” refers to a value that is within 10% above or below the value being described.

As used herein, any values provided in a range of values include both the upper and lower bounds, and any values contained within the upper and lower bounds.

The term “antibody” as referred to herein includes whole antibodies and any antigen binding fragment (i.e., “antigen-binding portion”) or single chain version thereof. An “antibody” refers, in one preferred embodiment, to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

As used herein, “effective treatment” refers to treatment producing a beneficial effect, e.g., amelioration of at least one symptom of a disease or disorder. A beneficial effect can take the form of an improvement over baseline, i.e., an improvement over a measurement or observation made prior to initiation of therapy according to the method. Effective treatment may refer to alleviation of at least one symptom of sickle cell disease.

The term “fused to” as used herein refers to a polypeptide made by combining more than one sequence, typically by cloning one sequence, e.g., a coding sequence, into an expression vector in frame with one or more second coding sequence(s) such that the two (or more) coding sequences are transcribed and translated into a single continuous polypeptide. In addition to being made by recombinant technology, parts of a polypeptide can be “fused to” each other by means of chemical reaction, or other means known in the art for making custom polypeptides.

A “heavy chain antibody” refers to an antibody that consists of two heavy chains and lacks the two light chains found in conventional antibodies. Camelids (members of the biological family Camelidae, the only currently living family in the suborder Tylopoda; extant camelids include dromedary camels, Bactrian camels, wild or feral camels, llamas, alpacas, vicunas and guanacos) are the only mammals with single chain VHH antibodies. About 50% of the antibodies in camelids are heavy chain antibodies with the other 50% being of the ordinary or conventional mammalian heavy/light chain antibody type.

“VHH domain” refers to variable domains present in naturally occurring heavy chain antibodies to distinguish them from the heavy chain variable domains that are present in conventional four chain antibodies (referred to herein as “VH domains”) and from the light chain variable domains that present in conventional four chain antibodies (referred to herein as “VL domains”).

VHH domains have a number of unique structural characteristics and functional properties that make isolated VHH domains (as well as sdAbs, which are based on VHH domains and share these structural characteristics and functional properties with the naturally occurring VHH domains) and proteins containing the VHH domains highly advantageous for use as functional antigen binding domains or proteins. For example, VHH domains, which bind to an antigen without the presence of a VL, and sdAbs can function as a single, relatively small, functional antigen binding structural unit, domain or protein. The small size of these molecules distinguishes VHH domains from the VH and VL domains of conventional four-chain antibodies. The use of VHH domains and sdAbs as single antigen-binding proteins or as antigen-binding domains (e.g., as part of a larger protein or polypeptide) offers a number of significant advantages over the use of conventional VH and VL domains, as well as scFv or conventional antibody fragments (such as Fab or F(ab′)2 fragments). Only a single domain is required to bind an antigen with high affinity and with high selectivity, for example, so that there is no need to have two separate domains present, nor to assure that these two domains are present in a particular spatial conformation and configuration (e.g., through the use of specific linkers, as with an scFv). VHH domains and sdAbs can also be expressed from a single gene and require no post-translational folding or modifications. VHH domains and sdAbs can easily be engineered into multivalent and multi-specific formats. VHH domains and sdAbs are also highly soluble and do not have a tendency to aggregate (Ward, E. et al., Nature, 341:544-6, 1989), and they are highly stable to heat, pH, proteases and other denaturing agents or conditions (Ewert, S. et al., Biochemistry, 41:3628-36, 2002). VHH domains and sdAbs are relatively easy and cheap to prepare, even on a scale required for production. For example, VHH domains, sdAbs, and polypeptides containing VHH domains or sdAbs can be produced using microbial fermentation using methods known in the art and do not require the use of mammalian expression systems, as with, for example, conventional antibody fragments. VHH domains and sdAbs are relatively small (approximately 15 kDa, or 10 times smaller than a conventional IgG) compared to conventional four-chain antibodies and antigen-binding fragments thereof, and therefore show higher penetration into tissues (including but not limited to solid tumors and other dense tissues) than conventional four-chain antibodies and antigen-binding fragments thereof. VHH domains and sdAbs can show so-called “cavity-binding” properties (due to, for example, their extended CDR3 loop) and can access targets and epitopes not accessible to conventional four-chain antibodies and antigen-binding fragments thereof. It has been shown, for example, that VHH domains and sdAbs can inhibit enzymes (WO 97/49805; Transue, T. et al., Proteins, 32:515-22, 1998; Lauwereys, M. et al., EMBO J., 17:3512-20, 1998).

The term “single-domain antibody,” or “sdAb,” as used herein, is an antibody or fragment thereof consisting of a single monomeric variable antibody domain. It is not limited to a specific biological source or to a specific method of preparation. A sdAb can be obtained, for example, by (1) isolating the VHH domain of a naturally occurring heavy chain antibody; (2) expressing a nucleotide sequence encoding a naturally occurring VHH domain; (3) “humanization” of a naturally occurring VHH domain or by expression of a nucleic acid encoding such humanized VHH domain; (4) “camelization” of a naturally occurring VH domain from any animal species, in particular a species of mammal, such as from a human being, or by expression of a nucleic acid encoding such a camelized VH domain; (5) “camelization” of a “domain antibody” (“Dab”) or by expression of a nucleic acid encoding such a camelized VH domain; (6) using synthetic or semi-synthetic techniques for preparing engineered polypeptides or fusion proteins; (7) preparing a nucleic acid encoding a sdAb using techniques for nucleic acid synthesis, followed by expression of the nucleic acid thus obtained; and/or (8) any combination of the above.

The fusion polypeptides or fusion proteins described herein can comprise, for example, amino acid sequences of naturally occurring VHH domains that have been “humanized,” e.g., by replacing one or more amino acid residues in the amino acid sequence of the naturally occurring VHH sequence by one or more of the amino acid residues that occur at the corresponding positions in a VH domain from a human being.

The fusion polypeptides or fusion proteins described herein can comprise, for example, amino acid sequences of naturally occurring VH domains that have been “camelized,” i.e., by replacing one or more amino acid residues in the amino acid sequence of a naturally occurring VH domain with one or more of the amino acid residues that occur at the corresponding positions in a VHH domain of, for example, a camelid antibody. This can be performed in a manner known in the art. Such camelization may preferentially occur at amino acid positions that are present at the VH-VL interface and at the so-called “Camelidae hallmark residues” (WO 94/04678). The VH domain or sequence that is used as a parental sequence or starting material for generating or designing the camelized sequence can be, for example, a VH sequence from a mammal, and in certain embodiments, the VH sequence of a human. It should be noted, however, that such camelized sequences can be obtained in any suitable manner known in the art and thus are not strictly limited to polypeptides that have been obtained using a polypeptide that comprises a naturally occurring parental VH domain.

Both “humanization” and “camelization” can be performed by providing a nucleotide sequence that encodes a naturally occurring VHH domain or V_{H} domain, respectively, and then changing, in a manner known to those skilled in the art, one or more codons in the nucleotide sequence such that the new nucleotide sequence encodes a humanized or camelized sequence, respectively. Also, based on the amino acid sequence or nucleotide sequence of a naturally occurring VHH domain or VH domain, a nucleotide sequence encoding a desired humanized or camelized sequence can be designed and synthesized de novo using techniques for nucleic acid synthesis known in the art, after which the nucleotide sequence thus obtained can be expressed in a manner known in the art.

The terms “antigen” or “antigen target,” as used herein, refer to a molecule or a portion of a molecule that is capable of being bound to by an antibody, one or more Ig binding domain, or other immunological binding moiety, including, for example, the engineered polypeptides or fusion polypeptides disclosed herein. An antigen is capable of being used in an animal to produce antibodies capable of binding to an epitope of that antigen. An antigen may have one or more epitopes.

The term “antigen-binding fragment” of an antibody (or simply “antibody fragment”), as used herein, refers to one or more fragments or portions of an antibody that retain the ability to specifically bind to an antigen. Such “fragments” are, for example between about 8 and about 1500 amino acids in length, suitably between about 8 and about 745 amino acids in length, suitably about 8 to about 300, for example about 8 to about 200 amino acids, or about 10 to about 50 or 100 amino acids in length. It has been shown that the antigen-binding function of an antibody can be performed by fragments of a full-length antibody. Examples of binding fragments encompassed within the term “antigen-binding fragment” of an antibody include (i) a Fab fragment, a monovalent fragment consisting of the V_{L}, V_{H}, CL and CH1 domains; (ii) a F(ab′)₂ fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR) or (vii) a combination of two or more isolated CDRs, which may optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (sFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

As used herein, the term “binding domain” refers to the portion of a protein or antibody that comprises the amino acid residues that interact with an antigen. Binding domains include, but are not limited to, antibodies (e.g., full length antibodies), as well as antigen-binding portions thereof. The binding domain confers on the binding agent its specificity and affinity for the antigen. The term also covers any protein having a binding domain that is homologous or largely homologous to an immunoglobulin-binding domain.

The term “epitope” or “antigenic determinant” refers to a site on an antigen to which an immunoglobulin or antibody specifically binds. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given antibody (i.e., epitope mapping) are well known in the art and include, for example, immunoblotting and immunoprecipitation assays, wherein overlapping or contiguous peptides from the antigen are tested for reactivity with the given antibody. Methods of determining spatial conformation of epitopes include techniques in the art and those described herein, for example, x-ray crystallography and 2-dimensional nuclear magnetic resonance (see, e.g., Epitope Mapping Protocols in Methods in Molecular Biology, Vol. 66, G. E. Morris, Ed. (1996)).

The term “bispecific” refers to a fusion polypeptide of the disclosure that is capable of binding two antigens.

The term “effective amount” refers to an amount of an agent that provides the desired biological, therapeutic and/or prophylactic result. That result can be reduction, amelioration, palliation, lessening, delaying and/or alleviation of one or more of the signs, symptoms or causes of a disease, or any other desired alteration of a biological system. In one example, an “effective amount” is the amount of fusion polypeptide or fragment thereof clinically proven to alleviate at least one symptom of sickle cell disease. An effective amount can be administered in one or more administrations.

As used herein, “effective treatment” refers to treatment producing a beneficial effect, e.g., amelioration of at least one symptom of a disease or disorder. A beneficial effect can take the form of an improvement over baseline, i.e., an improvement over a measurement or observation made prior to initiation of therapy according to the method. Effective treatment may refer to alleviation of at least one symptom of sickle cell disease.

The term “fused to” as used herein refers to a polypeptide made by combining more than one sequence, typically by cloning one sequence, e.g., a coding sequence, into an expression vector in frame with one or more second coding sequence(s) such that the two (or more) coding sequences are transcribed and translated into a single continuous polypeptide. In addition to being made by recombinant technology, parts of a polypeptide can be “fused to” each other by means of chemical reaction, or other means known in the art for making custom polypeptides.

The term “peptide linker” as used herein refers to one or more amino acid residues inserted or included between the engineered polypeptides of the fusion polypeptide(s). The peptide linker can be, for example, inserted or included at the transition between the engineered polypeptides of the fusion polypeptide at the sequence level.

The terms “pharmaceutical composition” or “therapeutic composition,” as used herein, refer to a compound or composition capable of inducing a desired therapeutic effect when administered to a patient.

The term “pharmaceutically acceptable carrier” or “physiologically acceptable carrier,” as used herein, refers to one or more formulation materials suitable for accomplishing or enhancing the delivery of the engineered polypeptides or fusion polypeptides of the disclosure.

An antibody, immunoglobulin, or immunologically functional immunoglobulin fragment, or the engineered polypeptides or fusion polypeptides disclosed herein, are said to “specifically” bind an antigen when the molecule preferentially recognizes its antigen target in a complex mixture of proteins and/or macromolecules. The term “specifically binds,” as used herein, refers to the ability of an antibody, immunoglobulin, or immunologically functional immunoglobulin fragment, or an engineered polypeptide or fusion polypeptide of the disclosure, to bind to an antigen containing an epitope with an K_{D} of at least about 10⁻⁶ M, 10⁻⁷ M, 10⁻⁸ M, 10⁻⁹ M, 10⁻¹⁰ M, 10⁻¹¹ M, 10⁻¹² M, or more, and/or to bind to an epitope with an affinity that is at least two-fold greater than its affinity for a nonspecific antigen.

As used herein, the term “subject” or “patient” is a human patient (e.g., a patient having sickle cell disease). As used herein, the term “subject” and “patient” are interchangeable.

The terms “treatment” or “treat,” as used herein, refer to both therapeutic treatment and prophylactic or preventative measures. Those in need of treatment include those having the disorder as well as those at risk of having the disorder or those in which the disorder is to be prevented.

### DETAILED DESCRIPTION

The disclosure provides methods of treating sickle cell disease (SCD) in subjects in need thereof by administering a fusion polypeptide comprising an engineered polypeptide that specifically binds human properdin fused to a polypeptide that specifically binds to human serum albumin by way of a peptide linker.

Fusion Polypeptides that Specifically Bind Albumin and Properdin

Described herein are fusion proteins and formulations thereof. In some embodiments, the fusion protein is a bispecific antibody where two antigen binding polypeptides are linked (e.g., by a linker such as the linker). Such bispecific constructs may include an anti-properdin binding polypeptide (e.g., a monovalent VHH antibody, or VHH variable domain) connected by a linker to a second polypeptide (e.g., a second monovalent antibody or VHH variable domain). The second polypeptide can, for example, enhance in vivo stability of the bispecific construct, target a different therapeutic target, or place two antigens in close proximity (e.g., thereby targeting the first bound antigen to the second bound antigen). In some embodiments, the second polypeptide is an albumin binding molecule, an albumin binding peptide, or an anti-albumin antibody (e.g., a monovalent antibody), or a modified form thereof (e.g., the variable domain of a llama antibody that specifically binds to human serum albumin). In addition to the present disclosure, albumin binding peptides are known in the art (WO 2007/106120 (see Tables 1 to 9); Dennis, M. et al., J. Biol. Chem., 277:35035-43, 2002; the disclosures of which are hereby incorporated by reference).

The antibodies described herein can inhibit, for example, properdin binding to C3b, C3Bb, and C3bBb. Inhibition of properdin leads to reduced alternative pathway complement activation, indicating a therapeutic benefit for patients afflicted with a disease of alternative pathway dysregulation wherein the alternative pathway is hyper-activated.

Anti-properdin antibodies described herein can be produced by using full-length properdin, properdin polypeptides, and/or using antigenic properdin epitope-bearing peptides, for example, a fragment of the properdin polypeptide. Properdin peptides and polypeptides can be isolated and used to generate antibodies as natural polypeptides, recombinant or synthetic recombinant polypeptides. All antigens useful for producing anti-properdin antibodies can be used to generate monovalent antibodies. Suitable monovalent antibody formats, and methods for producing them, are known in the art (WO 2007/048037 and WO 2007/059782, the entire contents of which are incorporated herein by reference).

The anti-properdin antibody may be a monoclonal antibody or derived from a monoclonal antibody. Suitable monoclonal antibodies to selected antigens may be prepared by known techniques (“Monoclonal Antibodies: A manual of techniques,” Zola (CRC Press, 1988); “Monoclonal Hybridoma Antibodies: Techniques and Applications,” Hurrell (CRC Press, 1982), the entire contents of which are incorporated herein by reference).

In other embodiments, the antibody may be a single-domain antibody, such as a VHH. Such antibodies exist naturally in camelids and sharks (Saerens, D. et al., Curr. Opin. Pharmacol., 8:600-8, 2008). Camelid antibodies are described in, for example, U.S. Pat. Nos. 5,759,808; 5,800,988; 5,840,526; 5,874,541; 6,005,079; and 6,015,695, the entire contents of each of which are incorporated herein by reference. The cloned and isolated VHH domain is a stable polypeptide that features the full antigen-binding capacity of the original heavy-chain antibody. VHH domains, with their unique structural and functional properties, combine the advantages of conventional antibodies (high target specificity, high target affinity and low inherent toxicity) with important features of small molecule drugs (the ability to inhibit enzymes and access receptor clefts). Furthermore, they are stable, have the potential to be administered by means other than injection, are easier to manufacture, and can be humanized (U.S. Pat. Nos. 5,840,526; 5,874,541; 6,005,079, 6,765,087; EP 1589107; WO 97/34103; WO 97/49805; U.S. Pat. Nos. 5,800,988; 5,874,541 and 6,015,695, the entire contents of each of which are incorporated herein by reference).

The anti-properdin component of the bispecific antibodies described herein CDR sequences including CDR-H1 having an amino acid sequence that is at least 90% identical to GRISSIIHMA (SEQ ID NO: 2); CDR-H2 having an amino acid sequence that is at least 90% identical (e.g., at least 95% 96%, 97%, 98%, 99%, or 100%) to RVGTTVYADSVKG (SEQ ID NO: 3); and having an amino acid sequence that is at least 90% identical (e.g., at least 95%, 96%, 97%, 98%, 99%, or 100%) to LQYEKHGGADY (SEQ ID NO: 4). The bispecific antibodies described herein may include CDR-H1 having an amino acid sequence of GRISSIIHMA (SEQ ID NO: 2); CDR-H2 having an amino acid sequence of RVGTTVYADSVKG (SEQ ID NO: 3); and CDR-H3 having an amino acid sequence of LQYEKHGGADY (SEQ ID NO: 4).

Furthermore, the engineered fusion proteins described herein can specifically bind serum albumin in such a way that, when the engineered protein is bound to or otherwise associated with a serum albumin molecule, the binding of the serum albumin molecule to FcRn is not significantly reduced or inhibited as compared to the binding of the serum albumin molecule to FcRn when the polypeptide is not bound thereto. In this embodiment, “not significantly reduced or inhibited” means that the binding affinity for serum albumin to FcRn (as measured using a suitable assay, such as, for example, SPR) is not reduced by more than 50%, or by more than 30%, or by more than 10%, or by more than 5%, or not reduced at all. In this embodiment, “not significantly reduced or inhibited” also means that the half-life of the serum albumin molecule is not significantly reduced. In particular, the engineered polypeptides can bind to amino acid residues on serum albumin that are not involved in binding of serum albumin to FcRn. More particularly, engineered polypeptides can bind to amino acid residues or sequences of serum albumin that do not form part of domain III of serum albumin, e.g., engineered polypeptides that are capable of binding to amino acid residues or sequences of serum albumin that form part of domain I and/or domain II.

The anti-albumin component of the bispecific antibodies described herein can comprise CDR sequences including CDR-H1 having an amino acid sequence that is at least 87% identical to GRPVSNYA (SEQ ID NO: 5); CDR-H2 having an amino acid sequence that is at least 87% identical to INWQKTAT (SEQ ID NO: 6); and having an amino acid sequence that is at least 90% identical (e.g., at least 95%, 96%, 97%, 98%, 99%, or 100%) to AAVFRVVAPKTQYDYDY (SEQ ID NO: 7). The bispecific antibodies described herein may include CDR-H1 having an amino acid sequence of GRPVSNYA (SEQ ID NO: 5); CDR-H2 having an amino acid sequence of INWQKTAT (SEQ ID NO: 6); and CDR-H3 having an amino acid sequence of AAVFRVVAPKTQYDYDY (SEQ ID NO: 7).

In some embodiments, the fusion protein includes an anti-properdin binding portion and an anti-albumin binding portion. In some embodiments, where the anti-properdin binding domain has an exposed N-terminus, the N-terminal glutamine can convert into the cyclized pyro-glutamate. Such modifications are known in the art (see, e.g., Liu et al., The Journal of Biological Chemistry 286(13:11211-11217, 2011). The portion encoding the anti-properdin binding portion may have at least 90% (e.g., 95%, 96%, 97%, 98%, 99%, or 100%) identity to the amino acid sequence of:

(SEQ ID NO: 55)
EVQ LLESGGGLVQ PGGSLRLSCA ASGRISSIIH MAWFRQAPGK
ERELVSEISR VGTTVYADSV KGRFTISRDN SKNTLYLQMN
SLKPEDTAVY YCNALQYEKH GGADYWGQGT LVTVSS.

In some embodiments, the anti-properdin binding portion of the fusion protein includes SEQ ID NO: 55.

The portion encoding the anti-albumin binding portion may have at least 90% (e.g., 95%, 96%, 97%, 98%, 99%, or 100%) identity to the amino acid sequence of:

(SEQ ID NO: 56)
QVQLVESGGG LVKPGGSLRL SCAASGRPVS NYAAAWFRQA
PGKEREFVSA INWQKTATYA DSVKGRFTIS RDNAKNSLYL
QMNSLRAEDT AVYYCAAVFR VVAPKTQYDY DYWGQGTLVT
VSS.

In some embodiments, the anti-properdin binding portion of the fusion protein includes SEQ ID NO: 56.

In some embodiments, the fusion protein is encoded by a nucleic acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) identity to the nucleic sequence of:

(SEQ ID NO: 57)
CAGGTGCAGCTGGTGGAAAGCGGCGGAGGCCTGGTCAAGCCTGGCGGCAG
CCTGAGACTGAGCTGTGCCGCCAGCGGCAGACCCGTGTCCAATTACGCCG
CTGCCTGGTTCCGGCAGGCCCCTGGCAAAGAGAGAGAGTTCGTCAGCGCC
ATCAACTGGCAGAAAACCGCCACCTACGCCGACAGCGTGAAGGGCCGGTT
CACCATCAGCCGGGACAACGCCAAGAACAGCCTGTACCTGCAGATGAACT
CCCTGCGGGCCGAGGACACCGCCGTGTACTACTGCGCCGCTGTGTTCCGG
GTGGTGGCCCCCAAGACCCAGTACGACTACGATTACTGGGGCCAGGGCAC
CCTGGTCACCGTGTCATCTGGCGGAGGGGGAGAAGGCGGGGGAGGGGAAG
GGGGAGGCGGCGAAGTCCAGCTGCTGGAATCTGGGGGCGGACTGGTGCAG
CCAGGCGGCTCCCTCAGACTGTCTTGCGCCGCCTCCGGCCGGATCAGCAG
CATCATCCACATGGCCTGGTTTAGACAGGCTCCCGGAAAAGAACGCGAGC
TGGTGTCCGAGATCTCCAGAGTGGGCACCACCGTGTATGCCGACTCCGTG
AAAGGCAGATTCACAATCTCCCGCGACAACAGCAAGAATACTCTGTATCT
CCAGATGAATAGCCTGAAGCCCGAAGATACAGCCGTCTACTATTGCAACG
CCCTGCAGTACGAGAAGCACGGCGGAGCCGACTATTGGGGACAGGGAACA
CTCGTGACAGTGTCTAGCTGATGA.

In some embodiments, the fusion protein is encoded by a nucleic acid sequence having at least 90% (e.g., at least 91%, 92%, 93%, 94%, 95%, 96%, 97%, 98%, 99%, or 100%) identity to the nucleic sequence of SEQ ID NO: 57. In some embodiments, the fusion protein is encoded by a nucleic acid sequence of SEQ ID NO: 57.

In some embodiments, the fusion protein has an amino acid sequence having at least 90% identity (e.g., 91%, 92%, 93%, 94%, 95%, 96%, 97%, 98%, 99%, or 100%) to the amino acid sequence:

(SEQ ID NO: 1)
QVQLVESGGG LVKPGGSLRL SCAASGRPVS NYAAAWFRQA
PGKEREFVSA INWQKTATYA DSVKGRFTIS RDNAKNSLYL
QMNSLRAEDT AVYYCAAVFR VVAPKTQYDY DYWGQGTLVT
VSSGGGGEGGGGEGGGGEVQ LLESGGGLVQ PGGSLRLSCA
ASGRISSIIH MAWFRQAPGK ERELVSEISR VGTTVYADSV
KGRFTISRDN SKNTLYLQMN SLKPEDTAVY YCNALQYEKH
GGADYWGQGT LVTVSS.

In some embodiments, the fusion protein has an amino acid sequence having at least 95% identity (e.g., 95%, 96%, 97%, 98%, 99%, or 100%) to SEQ ID NO: 1. In some embodiments, the fusion protein has an amino acid sequence of SEQ ID NO: 1.

In some embodiments, the C-terminal residue of the properdin-binding domain of the fusion protein can be fused either directly or via a linker to the N-terminal residue of the human serum albumin binding domain. In other embodiments, the C-terminal residue of the complement component human serum albumin binding domain of the fusion protein can be fused either directly or via a peptide to the N-terminal residue of the properdin binding domain. The fusion proteins described herein may include one or more modified amino acid residues. For example, the amino acid sequence of SEQ ID NO: 1 may include one or more amino acid modifications. The amino acid modifications described herein include all amino acid modifications known in the art (see, e.g., Liu et al., The Journal of Biological Chemistry 286(13:11211-11217, 2011 and Manning et al., Pharmaceutical Research 27(4):544-575, 2010). In all contexts, known conversions of specific amino acids, e.g., during processing or purification of the fusion polypeptide, are to be included, e.g., conversion of an exposed N-terminal glutamine to pyro-glutamate.

### Linkers

As described herein, a linker is used to describe a linkage or connection between polypeptides or protein domains and/or associated non-protein moieties. In some embodiments, a linker is a linkage or connection between at least two polypeptide constructs, e.g., such that the two polypeptide constructs are joined to each other in tandem series (e.g., a monovalent antibody linked to a second polypeptide or monovalent antibody). A linker can attach the N-terminus or C-terminus of one antibody construct to the N-terminus or C-terminus of a second polypeptide construct.

Described herein are fusion proteins that comprise engineered proteins that specifically bind albumin and properdin, wherein the engineered proteins are fused directly or are linked via one or more suitable linkers or spacers. A peptide linker can be, for example, inserted or included at the transition between the engineered proteins of the fusion protein at the sequence level. The identity and sequence of amino acid residues in the linker may vary depending on the desired secondary structure.

A linker can be a simple covalent bond, e.g., a peptide bond, a synthetic polymer, e.g., a polyethylene glycol (PEG) polymer, or any kind of bond created from a chemical reaction, e.g., chemical conjugation. In the case that a linker is a peptide bond, the carboxylic acid group at the C-terminus of one protein domain can react with the amino group at the N-terminus of another protein domain in a condensation reaction to form a peptide bond. Specifically, the peptide bond can be formed from synthetic means through a conventional organic chemistry reaction well-known in the art, or by natural production from a host cell, wherein a polynucleotide sequence encoding the DNA sequences of both proteins, e.g., two antibody constructs, in tandem series can be directly transcribed and translated into a contiguous polypeptide encoding both proteins by the necessary molecular machineries, e.g., DNA polymerase and ribosome, in the host cell.

In the case that a linker is a synthetic polymer, e.g., a PEG polymer, the polymer can be functionalized with reactive chemical functional groups at each end to react with the terminal amino acids at the connecting ends of two proteins.

In the case that a linker (except peptide bond mentioned above) is made from a chemical reaction, chemical functional groups, e.g., amine, carboxylic acid, ester, azide, or other functional groups commonly used in the art, can be attached synthetically to the C-terminus of one protein and the N-terminus of another protein, respectively. The two functional groups can then react through synthetic chemistry means to form a chemical bond, thus connecting the two proteins together. Such chemical conjugation procedures are routine for those skilled in the art.

As described herein, a linker between two peptide constructs can be an amino acid linker including from 1-200 (e.g., 1-4, 1-10, 1-20, 1-30, 1-40, 2-10, 2-12, 2-16, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 150, 200) amino acids. Suitable peptide linkers are known in the art, and include, for example, peptide linkers containing flexible amino acid residues such as glycine and serine).

Glycine, serine, and alanine are useful for linkers having maximum flexibility. Any amino acid residue can be considered as a linker in combination with one or more other amino acid residues, which may be the same as or different from the first amino acid residue, to construct larger peptide linkers as necessary depending on the desired properties. In other embodiments, the linker is GGGGEGGGGEGGGGE (SEQ ID NO:10). In other embodiments, the linker is GGGGSGGGGSGGGGS (SEQ ID NO:11). Additional peptide linkers suitable for use in creating fusion proteins described herein include, for example, G₄S (SEQ ID NO:12), (G₄S)₂ (SEQ ID NO:13), (G₄S)₃ (SEQ ID NO:14), (G₄S)₄ (SEQ ID NO:15), (G₄S)₅ (SEQ ID NO:16), (G₄S)₆ (SEQ ID NO:17), (EAAAK)₃ (SEQ ID NO:18), PAPAP (SEQ ID NO:19), G₄SPAPAP (SEQ ID NO:20), PAPAPG₄S (SEQ ID NO:21), (GGGDS)₂ (SEQ ID NO:22), (GGGES)₂ (SEQ ID NO:23), GGGDSGGGGS (SEQ ID NO:24), GGGASGGGGS (SEQ ID NO:25), GGGESGGGGS (SEQ ID NO:26), ASTKGP (SEQ ID NO:27), ASTKGPSVFPLAP (SEQ ID NO:28), G₃P (SEQ ID NO:29), G₇P (SEQ ID NO:30), PAPNLLGGP (SEQ ID NO:31), G₆ (SEQ ID NO:32), G₁₂ (SEQ ID NO:33), APELPGGP (SEQ ID NO:34), SEPQPQPG (SEQ ID NO:35), (G₃S₂)₃ (SEQ ID NO:36), GGGGGGGGGSGGGS (SEQ ID NO:37), GGGGSGGGGGGGGGS (SEQ ID NO:38), (GGSSS)₃ (SEQ ID NO:39), (GS₄)₃(SEQ ID NO:40), G₄A(G₄S)₂ (SEQ ID NO:41), G₄SG₄AG₄S (SEQ ID NO:42), G₃AS(G₄S)₂ (SEQ ID NO:43), G₄SG₃ASG₄S (SEQ ID NO:44), G₄SAG₃SG₄S (SEQ ID NO:45), (G₄S)₂AG₃S (SEQ ID NO:46), G₄SAG₃SAG₃S (SEQ ID NO:47), G₄D(G₄S)₂ (SEQ ID NO:48), G₄SG₄DG₄S (SEQ ID NO:49), (G₄D)₂G₄S (SEQ ID NO:50), G₄E(G₄S)₂ (SEQ ID NO:51), G₄SG₄EG₄S (SEQ ID NO:52), (G₄E)₂G₄S (SEQ ID NO:53), and GGGGAGGGGAGGGGS (SEQ ID NO: 54). One of skill in the art can select a linker, for example, to reduce or eliminate post-translational modification, e.g., glycosylation, e.g., xylosylation. In certain embodiments, the fusion protein comprises at least two sdAbs, Dabs, VHH antibodies, VHH antibody fragments, or combination thereof wherein at least one of the sdAbs, Dabs, VHH antibodies, or VHH antibody fragments is directed against albumin and one of the sdAbs, Dabs, VHH antibodies, or VHH antibody fragments is directed against properdin, so that the resulting fusion protein is multivalent or multi-specific. The binding domains or moieties can be directed against, for example, HSA, cynomolgus monkey serum albumin, human properdin and/or cynomolgus monkey properdin.

### Methods of Treating Sickle Cell Disease

Provided herein are methods for treating sickle cell disease (SCD) in a human patient, comprising administering to the patient a fusion polypeptide comprising an engineered polypeptide that specifically binds human properdin fused to a polypeptide that specifically binds to human serum albumin by way of a peptide linker according to a particular clinical dosage regimen (i.e., at a particular dose amount and according to a specific dosing schedule).

In one embodiment, the fusion polypeptide is administered to the patient at a dose of 300 mg. In some embodiments, the fusion polypeptide is administered every week. The fusion polypeptide may be administered to the patient for between 1 and 13 weeks (e.g., between 1 week and 12 weeks, 1 week and 10 weeks, 1 week and 8 weeks, 1 week and 6 weeks, 1 week and 4 weeks, 4 weeks and 13 weeks, 6 weeks and 13 weeks, 8 weeks and 13 weeks, 10 weeks and 13 weeks, or 12 weeks and 13 weeks).

In some embodiments, the fusion polypeptide is administered once per week, twice per week, three times per week, four times per week, five times per week, six times per week, or daily. In another embodiment, the fusion polypeptide is administered twice daily. In another embodiment, the fusion polypeptide is administered once every two weeks, once every three weeks, once every four weeks, once every five weeks, once every six weeks, once every seven weeks, once every eight weeks, once every nine weeks, once every ten weeks, once every eleven weeks, or once every twelve weeks.

In another embodiment, the fusion polypeptide is administered to the patient every two weeks. (e.g., once every two weeks). The fusion polypeptide may be administered for up to 12 weeks. In some embodiments, the fusion polypeptide is administered up to six times (e.g., once, twice, three times, four times, five times, or six times). In some embodiments, the fusion polypeptide is administered up to four times (e.g., once, twice, three times, or four times).

In another embodiment, the fusion polypeptide is administered to the patient at a dose of 600 mg. In some embodiments, the fusion polypeptide is administered to the patient every 4 weeks. The fusion polypeptide may be administered the patient up to 4 times (e.g., once, twice, three times, or four times).

The patient who is administered the fusion polypeptide may have been clinically diagnosed with sickle cells disease by a clinician. The sickle cell disease may include HbSS or HbSβ⁰-thalassemia. The human patient may be between 18 and 65 years of age. In some embodiments, the human patient has a body weight of ≥40 kg. In some embodiments, the patient is further administered hydroxyurea. In some embodiments, the patient has been receiving a stable dose of hydroxyurea for at least 3 months prior to administration the antibody or the antigen binding fragment thereof. In another embodiment, the patient has not been administered hydroxyurea for at least 30 days prior to administration the antibody or the antigen binding fragment thereof.

In some embodiments, the fusion polypeptide is formulated for subcutaneous administration. For example, the fusion polypeptide may be formulated at a pH of 5.4 at a concentration of 150 mg/mL in an aqueous solution comprising 20 nM sodium acetate, 250 mM sucrose, and 0.05% polysorbate-80.

In some embodiments, the fusion polypeptide is administered using a pre-filled syringe. In other embodiments, the fusion polypeptide is administered using an autoinjector device. For example, the autoinjector device may include a single vial system, such as a pen injector device for solution delivery. Such devices are commercially available from manufacturers such as BD Pens, BD AutojectorO, Humaject®, NovoPen®, B-D®Pen, AutoPen®, and OptiPen®, GenotropinPen®, Genotronorm Pen®, Humatro Pen®, Reco-Pen®, Roferon Pen®, Biojector@, Iject®, J-tip Needle-Free Injector®, DosePro®, Medi-Ject®, e.g., as made or developed by Becton Dickinson (Franklin Lakes, NJ), Ypsomed (Burgdorf, Switzerland, www.ypsomed.com; Bioject, Portland, OR.; National Medical Products, Weston Medical (Peterborough, UK), Medi-Ject Corp (Minneapolis, MN), and Zogenix, Inc, Emeryville, CA. Recognized devices comprising a dual vial system include those pen-injector systems for reconstituting a lyophilized drug in a cartridge for delivery of the reconstituted solution such as the HumatroPen®. In one embodiment, the autoinjector is a YpsoMate 2.25 or YpsoMate 2.25 Pro (Ypsomed) disposable injection device.

In some embodiments, the patients treated according to the methods described herein have been vaccinated against meningococcal infections within three years prior to, or at the time of, initiating study drug. In one embodiment, patients who initiate treatment less than two weeks after receiving a meningococcal vaccine receive treatment with appropriate prophylactic antibiotics until two weeks after vaccination. In another embodiment, patients treated according to the methods described herein are vaccinated against meningococcal serotypes A, C, Y, W135 and/or B.

### Outcomes

In some embodiments, administration of any one of the fusion polypeptides described herein may result in the patient experience no treatment emergent adverse events after 12 weeks of treatment. In some embodiments, the patient experiences no serious adverse events after 12 weeks of treatment. In some embodiments, the patient experiences no adverse events after 12 weeks of treatment.

In some embodiments, a change in the serum concentration of the fusion polypeptide may be measured for up to 30 weeks after starting treatment. For example, a change in the serum concentration of the fusion polypeptide may be measured for up to 12 weeks after starting treatment. In some embodiments, a change in blood concentration of an anti-drug antibody may be measured for up to 30 weeks after starting treatment. For example, a change in blood concentration of an anti-drug antibody may be measured for up to 12 weeks after starting treatment. In some embodiments, a change in serum concentration from baseline of total and free properdin may be measured for after up to 30 weeks of treatment. For example, a change in serum concentration from baseline of total and free properdin may be measured for after up to 30 weeks of treatment.

In some embodiments, the patient experiences a change in serum concentration from baseline of complement component Ba (Ba), complement component C3a (C3a), or soluble complement component C5B-9 (sC5B9) after 12 weeks of treatment. In another embodiment, the patient may experience a change from baseline in blood or serum concentration of hemopexin, nitric oxide, an inflammatory marker, or a cell adhesion marker after 12 weeks of treatment. The inflammatory marker may be interleukin-1. The cell adhesion marker may be soluble P-selectin.

In some embodiments, after 12 weeks of treatment, the patient may experience a change from baseline in hemoglobin levels after 12 weeks of treatment. In another embodiment, the patient may experience a change in serum LDH levels, indirect bilirubin, haptoglobin, or hemopexin after 12 weeks from baseline. In another embodiment, the patient may experience a change in retinculocyte levels after 12 weeks from baseline

In some embodiments, the patient may experience a reduced rate of vaso-occlusive crisis after 12 weeks of treatment in comparison to baseline. In some embodiments, the patient experiences an increased time to first vaso-occlusive crisis after 12 weeks of treatment in comparison to baseline. The vaso-occlusive crisis may occur when the sickled red blood cells block blood flow to the point that tissues become deprived of oxygen, which may result in an inflammatory response as the body tries to rectify the problem, and may result in symptoms including pain, which can affect any part of the body, but most commonly occurs in the back, chest, or extremities.

### Kits and Unit Dosage Forms

Also provided herein are kits that include a pharmaceutical composition containing a fusion polypeptide described herein, such as the fusion polypeptide having the amino acid sequence of SEQ ID NO: 1, and a pharmaceutically acceptable carrier, in a therapeutically effective amount adapted for use in the preceding methods. The kits can also optionally include instructions, e.g., comprising administration schedules, to allow a practitioner (e.g., a physician, nurse or patient) to administer the composition contained therein to administer the composition to a patient having MG. The kit also can include a syringe.

Kits can optionally include multiple packages of the single-dose pharmaceutical compositions each containing an effective amount of the fusion polypeptide for a single administration in accordance with the methods provided above. Instruments or devices necessary for administering the pharmaceutical composition(s) also may be included in the kits. A kit may provide one or more pre-filled syringes containing an amount of the fusion polypeptide. A kit may include one or more autoinjectors containing an amount of the fusion polypeptide.

The following examples are merely illustrative and should not be construed as limiting the scope of this disclosure in any way as many variations and equivalents will become apparent to those skilled in the art upon reading the present disclosure. The contents of all references, Genbank entries, patents and published patent applications cited throughout this application are expressly incorporated herein by reference.

### EXAMPLES

The Examples that follow are illustrative of specific embodiments of the disclosure, and various uses thereof. They are set forth for explanatory purposes only, and they should not be construed as limiting the scope of the invention in any way.

### Example 1: A Phase 2a, Randomized, Open-Label Study to Evaluate Multiple Dosing Regimens of a Subcutaneous Anti-Properdin/Anti-Serum Albumin Bispecific Single Variable Domain Antibody in Adult Patients with Sickle Cell Disease

### 1.1 Study Rationale:

The fusion polypeptide described herein (anti-properdin/anti-serum albumin bispecific single variable domain on a heavy chain [VHH] antibody) is a novel properdin blocking agent being developed for the treatment of diseases involving dysregulated complement activity. The fusion polypeptide described herein molecule is bispecific, comprising a VHH antibody domain that binds and blocks properdin, connected via a linker to a VHH domain that binds serum albumin, thereby conferring an extended circulatory half-life to the molecule. The fusion polypeptide formulation described herein is designed for subcutaneous (SC) administration. The purpose of this study is to evaluate the safety, tolerability, efficacy, pharmacokinetics (PK), pharmacodynamics (PD), and immunogenicity of multiple doses and dosing regimens of the fusion polypeptide described herein SC in patients with Sickle Cell Disease (SCD).

The study includes up to 3 cohorts. Data from this study are anticipated to help design future studies in patients with SCD and other complement-mediated diseases.

### Objectives and Endpoints

TABLE 1
Mapping Objectives to Endpoints for Patients with Sickle Cell Disease

| Objectives | Endpoints |
|---|---|
| Primary | |
| To assess the safety and tolerability of fusion | Safety assessed by incidence of TEAEs and |
| polypeptide SC in patients with SCD | SAEs, physical examination, vital sign |
| | measurements, clinical laboratory, and |
| | electrocardiogram results through Day 211 |
| | (Cohorts 1 and 2) and through Day 169 |
| | (Optional Cohort 3) |
| Secondary | |
| To assess the multiple-dose PK of the fusion | Serum the fusion polypeptide described herein |
| polypeptide SC | multiple-dose PK profiles through Day 211 |
| | (Cohorts 1 and 2) and through Day 169 |
| | (Optional Cohort 3) |
| To assess the PD effects of the fusion | Change in serum concentrations of total and |
| polypeptide SC | free properdin over time through Day 211 |
| | (Cohorts 1 and 2) and through Day 169 |
| | (Optional Cohort 3) |
| | Change in CAP activity using the Wieslab AP |
| | assay through Day 211 (Cohorts 1 and 2) and |
| | through Day 169 (Optional Cohort 3) |
| To assess the effect of the fusion polypeptide | Change from baseline in complement |
| on complement biomarkers | biomarkers through Week 12 (Cohorts 1 and 2) |
| To assess the effect of the fusion polypeptide | Change from baseline in hemoglobin levels at |
| on hemolysis | Week 12 (Cohorts 1 and 2) |
| | Change from baseline in hemolysis markers |
| | (e.g., lactate dehydrogenase, reticulocytes, and |
| | bilirubin) and hemopexin at Week 12 (Cohorts 1 |
| | and 2) |
| To assess the immunogenicity of the fusion | Incidence of ADAs to the fusion polypeptide |
| polypeptide SC | described herein through Day 211 (Cohorts 1 |
| | and 2) and through Day 169 for Optional |
| | Cohort 3 |
| Exploratory | |
| To explore the effect of the fusion polypeptide | Change from baseline in VOC related |
| on vaso-occlusion markers | biomarkers through Week 12 (Cohorts 1 and 2) |
| To explore the clinical efficacy of the fusion | VOC events (rate of VOC, time to first VOC) |
| polypeptide SC in SCD | through Week 12 (Cohorts 1 and 2) |

Abbreviations: ADA = antidrug antibody; AP = alternative pathway; CAP = complement alternative pathway; PD = pharmacodynamics(s); PK = pharmacokinetic(s); SAE = serious adverse event; SC = subcutaneous; SCD = sickle cell disease; TEAE = treatment-emergent adverse event; VOC = vaso-occlusive crisis.

### 1.2 Overall Design

This is a Phase 2a study, with up to 3 multiple dose cohorts of open-label the fusion polypeptide described herein SC in adult patients with SCD (HbSS and HbSβ⁰-thalassemia).

The study is conducted in up to 30 adult patients with SCD enrolled in up to 3 open-label cohorts (Cohorts 1, 2 and 3 [optional]) to receive multiple SC doses of the open-label fusion polypeptide described herein. The fusion polypeptide described herein is administered as described in Table 2.

TABLE 2
Fusion Polypeptide Dosing Cohorts

| | | | | | Number |
|---|---|---|---|---|---|
| | | | Route of | | of Doses/ |
| | | | Adminis- | Planned | Dose |
| Cohort | N | Study Drug | tration | Dose | Interval |
| 1 | Up to 12 | The fusion | SC | 300 mg | QW × 13 |
| | maximum of 6 | polypeptide | | | |
| | on stable dose | described | | | |
| | of hydroxyurea | herein | | | |
| 2 | Up to 12 | The fusion | SC | 600 mg | Q4W × 4 |
| | maximum of 6 | polypeptide | | | |
| | on stable dose | described | | | |
| | hydroxyurea | herein | | | |
| 3 (op- | Up to 6 | The fusion | SC | 300 mg | Q2W × 4 |
| tional) | maximum of 3 | polypeptide | | | |
| | on stable dose | described | | | |
| | of hydroxyurea | herein | | | |

Abbreviations: N = number of participants; QW = once weekly; Q2W = once every 2 weeks; Q4W = once every 4 weeks; SC = subcutaneous; SCD = Sickle Cell Disease.

The dose and dosing interval for Cohorts 1 and 2 was determined using cumulative safety data, an interim PK/PD analysis from participants enrolled in the study described herein, and data from the 6-month Good Laboratory Practice (GLP) toxicology study in monkeys. Optional Cohort 3 is initiated after evaluation of safety and PK/PD data from Cohorts 1 and 2, at discretion of those evaluating the study.

Cohort 1 and Cohort 2 run in parallel, and patients are randomized 1:1 to either of the cohorts on determination of eligibility. The decision to initiate Optional Cohort 3 is made at the discretion of those performing the study and is based on analysis of PK/PD and safety after at least 8 patients from Cohorts 1 and 2 (4 from each cohort) have enrolled. Furthermore, enrollment of Optional Cohort 3 starts after Cohorts 1 and 2 are fully enrolled. Each cohort is stratified to ensure patients with SCD who are treated with a stable dose of hydroxyurea and patients with SCD who are not currently treated with hydroxyurea are included. The Treatment Period is 12 weeks for Cohorts 1 and 2, and 6 weeks for Cohort 3. For patients who were previously treated with hydroxyurea but are not currently on hydroxyurea, treatment must have been stopped at least 30 days prior to providing informed consent.

At the discretion of the Investigator, and after consultation with the Data Monitoring Committee (DMC), additional participants with SCD may be enrolled as replacement participants if a participant discontinues during the Dosing Period for reasons other than drug-related adverse events (AEs).

### Disclosure Statement

This is an open-label, parallel group intervention study with up to 3 treatment arms.

### Number of Participants:

Up to 30 adult patients with SCD (HbSS and HbSβ⁰-thalassemia) are enrolled in up to 3 cohorts and receive multiple open-label SC doses of the fusion polypeptide described herein. The cohorts enroll both patients with SCD who are being treated with a stable dose of hydroxyurea and patients with SCD who are not currently treated with hydroxyurea. For patients who were previously treated with but are not currently on hydroxyurea, treatment must have been stopped at least 30 days prior to providing informed consent.

### Intervention Groups and Duration:

The planned study duration is approximately 38 weeks for Cohorts 1 and 2: up to 56 days (8 weeks) for Screening, 84 days (12 weeks) for the Treatment Period, and 126 days (18 weeks) for the Follow-up Period. Patients attend outpatient visits during the Treatment and Follow-up Period with the option to stay at the inpatient facility. The end of study (EOS) for each individual patient is anticipated to be Day 211 (210 days) or the timepoint at which complement activity has returned to a normal range or 80% of baseline if later than Day 211.

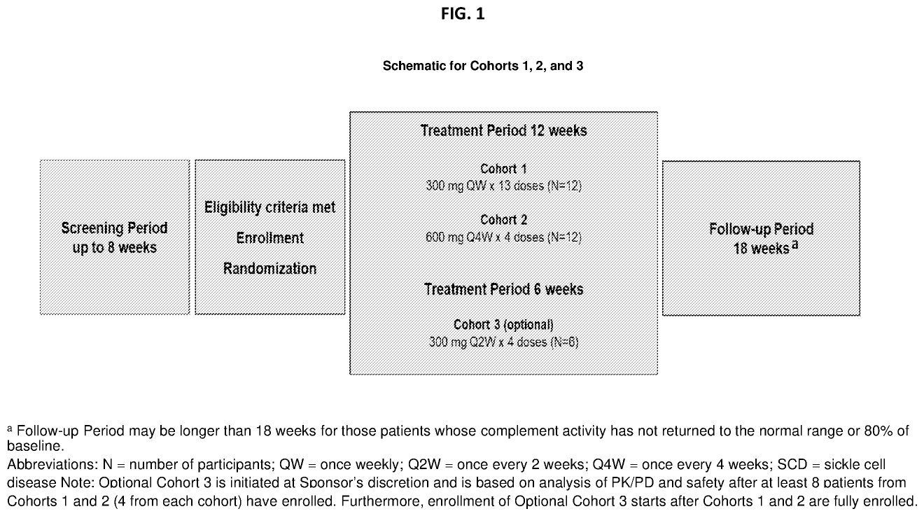

The planned study duration is approximately 32 weeks for optional Cohort 3: up to 56 days (8 weeks) for Screening, 42 days (6 weeks) for the Treatment Period, and 126 days (18 weeks) for the Follow-up Period. Patients attend outpatient visits during the Treatment and Follow-up Period with the option to stay at the inpatient facility. The EOS for each individual patient is anticipated to be Day 169 (168 days) or the timepoint at which complement activity has returned to a normal range or 80% of baseline if later than Day 169. A schematic view of the study is presented in FIG. 1.

Data Monitoring Committee: This study uses an independent DMC to monitor safety and to perform the planned interim analyses of the study.

### Statistical Analyses—All Cohorts:

### Populations for Analysis

For purposes of analysis, the following populations are defined:

| Population | Description |
|---|---|
| Safety | All participants who receive at least 1 dose of study drug |
| Enrolled Set | All participants who sign the ICF and passed inclusion/ |
| | exclusion criteria. |
| Full Analysis | All participants in the Enrolled Set who have been |
| Set (FAS) | randomized and received at least 1 dose of study drug. |
| Per Protocol | All participants in the Enrolled Set without any major |
| Set (PPS) | protocol deviation. |
| Pharma- | All participants who receive at least 1 dose of the study |
| cokinetic | drug and have at least 1 post-dose PK sample |
| Pharma- | All participants who receive at least 1 dose of study drug |
| codynamic | and who have evaluable properdin concentration data CAP |
| | or CCP activity data |
| Immuno- | All participants who have a predose and at least 1 postdose |
| genicity | ADA sample collected that can be analyzed |

Abbreviations: ADA = antidrug antibody; CAP = complement alternative pathway; CCP = complement classical pathway; ICF = informed consent form; PK = pharmacokinetic(s).

### Safety Analysis

All safety analyses are performed on the Safety Population and is reported by each cohort and treatment arm.

Safety analyses includes an analysis of all treatment-emergent adverse events (TEAEs), electrocardiograms (ECG's), clinical laboratory data, physical examinations, and vital sign measurements using descriptive statistics. No inferential statistical analyses are planned on the safety parameters of this study. The prevalence of AEs and serious adverse events (SAEs) is summarized, by System Organ Class (SOC) and Preferred Term for each cohort and treatment arm and overall, within each treatment arm, by relationship to study drug. AEs are summarized by cohort and overall by severity. SAEs and AEs resulting in withdrawal from the study are listed. Participants having multiple AEs within a category (e.g., overall, SOC, Preferred Term) are counted once in that category. For severity tables, a participant's most severe event within a category is counted.

All concomitant medications are coded using the World Health Organization Drug Dictionary, and the frequency and percentage of concomitant medications are summarized.

### Efficacy Analysis

Absolute and percentage change from baseline in complement biomarkers, hemoglobin and hemolysis markers are evaluated at the end of the Treatment Period (12 weeks) for Cohort 1 and Cohort 2. Time to hemoglobin response (defined as an increase in hemoglobin levels of >1 g/dL from baseline) is evaluated at the end of the Treatment Period (12 weeks) for Cohort 1 and Cohort 2.

### Pharmacokinetic Analysis

The individual serum concentration data from participants who receive the SC dose of the fusion polypeptide described herein with actual sampling dates and times, is used to characterize the PK by population PK analysis approach.

### Pharmacodynamic Analysis

The PD effects of all the SC doses of the fusion polypeptide described herein administered is evaluated by assessing changes in serum total and free properdin concentrations and complement alternative pathway (CAP) activity using the Wieslab alternative pathway (AP) assay. In addition, complement classical pathway activity and other measures of properdin activity over time may be considered as deemed appropriate (Section 8.6).

### Immunogenicity Analysis

Immunogenicity, as measured by incidence of antidrug antibody (ADA) to the fusion polypeptide described herein are summarized.

### Exploratory Analysis

Additional exploratory analyses on biomarker assays and clinical efficacy endpoints may be conducted. Details of these analyses are presented in the statistical analysis plan (SAP).

### Interim Analysis

An interim analysis may be performed after at least 12 patients from Cohorts 1 and 2 (6 from each cohort) have enrolled and completed the Treatment Period (12 weeks). Details of this analysis are presented in the SAP.

### 1.3 Schedule of Activities (SoA)

The SoA for once weekly (QW) dosing for multiple-dose Cohort 1 for Screening through Day 85 is presented in Table 3.

The SoA for once every 4 weeks (Q4W) dosing for multiple-dose Cohort 2 for Screening through Day 85 is presented in Table 4.

Table 5 presents the SoA for intensive PK/PD sampling for a subset of participants in Cohorts 1 and 2. Table 6 presents the SoA for Cohorts 1 and 2 for Day 99 through the end of the Follow-up Period.

The SoA for once every 2 weeks (Q2W) dosing for optional multiple-dose Cohort 3 for Screening through Day 43 is presented in Table 7. Table 8 presents the SoA for Cohort 3 for Day 57 through the end of the Follow-up Period.

TABLE 3
Schedule of Activities-Screening Through Day 85 (Cohort 1)-QW Dosing

| | Study Day | | | | | | | | | | | | | |
|---|---|---|---|---|---|---|---|---|---|---|---|---|---|---|
| | Screening | | | | | | | | | | | | | |
| | Day −56 | | | | | | | | | | | | | |
| | to Day | Day | Day | Day | Day | Day | Day | Day | Day | Day | Day | Day | Day | Day |
| | −1 | 1 | 8 | 15 | 22 | 29 | 36 | 43 | 50 | 57 | 64 | 71 | 78 | 85 |
| | Window (Day) | | | | | | | | | | | | | |
| | NA | NA | ±1 | ±1 | ±1 | ±1 | ±1 | ±1 | ±1 | ±1 | ±1 | ±1 | ±1 | ±1 |
| Informed consent^{a} | X | | | | | | | | | | | | | |
| Eligibility criteria^{b} | X | X | | | | | | | | | | | | |
| Randomization^{c} | | X | | | | | | | | | | | | |
| Confirmation of or | X | | | | | | | | | | | | | |
| administration of | | | | | | | | | | | | | | |
| meningococcal | | | | | | | | | | | | | | |
| vaccination^{d} | | | | | | | | | | | | | | |
| Confirmation of or | X | | | | | | | | | | | | | |
| administration of | | | | | | | | | | | | | | |
| HiB and | | | | | | | | | | | | | | |
| S pneumoniae | | | | | | | | | | | | | | |
| vaccination | | | | | | | | | | | | | | |
| Medical history and | X | | | | | | | | | | | | | |
| demographics | | | | | | | | | | | | | | |
| Physical | X | X | | | | | | | | | | | | |
| examination | | | | | | | | | | | | | | |
| Abbreviated | | | | | | X | | | | X | | | | X |
| physical | | | | | | | | | | | | | | |
| examination | | | | | | | | | | | | | | |
| Height, weight, and | X | X | | | | X | | | | X | | | | X |
| BMI^{e} | | | | | | | | | | | | | | |
| Hematology^{f} | X | X | | | | X | | | | X | | | | X |
| Clinical chemistry^{f} | X | X | | | | X | | | | X | | | | X |
| Coagulation^{f} | X | X | | | | X | | | | X | | | | X |
| Hepatitis B and C | X | | | | | | | | | | | | | |
| screen | | | | | | | | | | | | | | |
| HIV (types 1 and 2) | X | | | | | | | | | | | | | |
| screen | | | | | | | | | | | | | | |
| Serum/urine | X | X | | | | X | | | | X | | | | X |
| pregnancy test^{g} | | | | | | | | | | | | | | |
| Urinalysis | X | X | | | | X | | | | X | | | | X |
| (including spot | | | | | | | | | | | | | | |
| urine) | | | | | | | | | | | | | | |
| Vital sign | X | X | X | X | X | X | X | X | X | X | X | X | X | X |
| measurements^{h} | | | | | | | | | | | | | | |
| Triplicate ECGⁱ | X | X | | X | | X | | | | X | | | | X |
| Study drug | | X | X | X | X | X | X | X | X | X | X | X | X | X |
| administration QW | | | | | | | | | | | | | | |
| PK samples^{j} | | X | | | | | | X | | X | | X | | X |
| Blood sample for | | X | | X | | X | | X | | X | | X | | X |
| PD panel^{j, k} | | | | | | | | | | | | | | |
| Injection site | | X | X | X | X | X | X | X | X | X | X | X | X | X |
| reaction^{l} | | | | | | | | | | | | | | |
| Biomarkers^{m} | X | X | | X | | X | | X | | X | | X | | X |
| Immunogenicity | | X | | X | | X | | | | | | | | X |
| (fusion polypeptide | | | | | | | | | | | | | | |
| ADA)ⁿ | | | | | | | | | | | | | | |
| Assessment of | X | ←Monitor continuously→ | | | | | | | | | | | | |
| VOC^{o} | | | | | | | | | | | | | | |
| Review patient | X | X | X | X | X | X | X | X | X | X | X | X | X | X |
| diary^{p} | | | | | | | | | | | | | | |
| Review safety card^{q} | X | X | X | X | X | X | X | X | X | X | X | X | X | X |
| Concomitant | ←Monitor continuously (after ICF is signed)→ | | | | | | | | | | | | | |
| medications | | | | | | | | | | | | | | |

| | |
|---|---|
| Adverse events^{r} | ←Monitor continuously (after ICF is signed)→ |

^{a}Signed and dated IEC-approved informed consent must be obtained before any study-specific screening procedures are performed.
^{b}Eligibility is assessed at Screening and Day 1.
^{c}Randomization occurs following confirmation of all eligibility requirements.
^{d}To reduce the risk of meningococcal infection (Neisseria meningitidis), all patients are vaccinated with MCV4 and serogroup B meningococcal vaccinations at least 14 days before dosing, if not already vaccinated within 3 years before the first dose (or per national/local guidelines). Participants who initiate study intervention treatment less than 14 days after receiving a meningococcal vaccine must receive treatment with appropriate prophylactic antibiotics until at least 2 weeks after vaccination. Every effort should be made to start the meningococcal vaccination series at least 14 days prior to randomization.
^{e}Height and BMI only at Screening.
^{f}Non-fasting blood samples are obtained.
^{g}Serum pregnancy test is required for all female participants of childbearing potential (i.e., have achieved menarche) to confirm that a female participant is not pregnant prior to first dosing. A urine pregnancy test can be performed if the Screening visit is within 3 weeks of Day 1 visit. A urine (or serum if required by local site policy) pregnancy test is to be performed at all other required timepoints.
^{h}At Screening, supine and standing (orthostatic) blood pressures are performed.
ⁱPredose triplicate 12-lead ECGs are performed on Day 1 only and is performed 3 times at approximately 2, 1, and 0.5 hours before the start of dosing (If this schedule is not feasible, ECGs performed with at least 20 min apart is acceptable). If the scheduled dosing is delayed, predose ECGs performed within 4 hours of dosing do not need to be repeated. Postdose ECGs is performed within 30 min before PK/PD sample collection.
^{j}PK and PD samples are collected at pre-dose and between 2 and 4 hours post dose.
^{k}PD samples are collected to assess serum total and free properdin and CAP activity.
^{l}Injection site reaction evaluation is conducted on dosing days and if a reaction occurs, the evaluation continues until the reaction is fully resolved.
^{m}Blood samples for biomarkers is collected 2 times prior to receiving the first dose (collect once during Screening and then again at Day 1 pre-dose, the 2 collections should be at least 2 weeks apart). Collection of blood (for serum, citrated and K2—EDTA, or P100 plasma) and urine.
ⁿIn case of any suspected case of hypersensitivity or anaphylaxis additional samples for the assessment of ADA may be collected at or near the time of the event.
^{o}If a VOC occurs, samples is collected as per unscheduled visit.
^{p}The patient records symptoms of VOC in a paper diary throughout the study. The Investigator or qualified designee reviews the patient diary at every visit.
^{q}The Investigator or qualified designee meets with the participant at each visit to ensure they carry the clinical study participant safety card at all times and to review the potential meningococcal infection risks of the fusion polypeptide described herein, and to address any safety concerns of the participant.
^{r}Collection of adverse events and serious adverse events begins after ICF signing.
Abbreviations: ADA = antidrug antibody; BMI = body mass index; CAP = complement alternative pathway; ECG = electrocardiogram; HiB = Haemophilus influenzae type B; HIV = human immunodeficiency virus; ICF = informed consent form; IEC = Independent Ethics Committee; K2—EDTA = dipotassium ethylenediaminetetraacetic acid; MCV4 = tetravalent meningococcal conjugate vaccine; NA = not applicable; PD = pharmacodynamic(s); PK = pharmacokinetic(s); QW = once weekly; VOC = vaso-occlusive crisis.

TABLE 4
Schedule of Activities-Screening Through Day 85 (Cohort 2)-Q4W Dosing

| | Study Day | | | | | | | |
|---|---|---|---|---|---|---|---|---|
| | Screening | | | | | | | |
| | Day −56 to | | | | | | | |
| | Day −1 | Day 1 | Day 15 | Day 29 | Day 43 | Day 57 | Day 71 | Day 85 |
| | Window (Day) | | | | | | | |
| | NA | NA | ±3 | ±3 | ±3 | ±3 | ±3 | ±3 |
| Informed consent^{a} | X | | | | | | | |
| Eligibility criteria^{b} | X | X | | | | | | |
| Randomization^{c} | | X | | | | | | |
| Confirmation of or | X | | | | | | | |
| administration of | | | | | | | | |
| meningococcal | | | | | | | | |
| vaccination^{d} | | | | | | | | |
| Confirmation of or | X | | | | | | | |
| administration of HiB and | | | | | | | | |
| S pneumoniae vaccination | | | | | | | | |
| Medical history and | X | | | | | | | |
| demographics | | | | | | | | |
| Physical examination | X | X | | | | | | |
| Abbreviated physical | | | | X | | X | | X |
| examination | | | | | | | | |
| Height, weight, and BMI^{e} | X | X | | X | | X | | X |
| Hematology^{f} | X | X | | X | | × | | X |
| Clinical chemistry^{f} | X | X | | X | | X | | X |
| Coagulation^{f} | X | X | | X | | X | | X |
| Hepatitis B and C screen | X | | | | | | | |
| HIV (types 1 and 2) screen | X | | | | | | | |
| Serum/urine pregnancy | X | X | | X | | X | | X |
| test^{g} | | | | | | | | |
| Urinalysis (including spot | X | X | | X | | X | | X |
| urine) | | | | | | | | |
| Vital sign measurements^{h} | X | X | X | X | X | X | X | X |
| Triplicate ECGⁱ | X | X | X | X | | X | | X |
| Study drug administration | | X | | X | | X | | X |
| Q4W | | | | | | | | |
| PK samples^{j} | | X | X2 | X | X | X | X | X |
| Blood sample for PD | | X | X | X | X | X | X | X |
| panel^{j, k} | | | | | | | | |
| Injection site reaction^{l} | | X | X | X | X | X | X | X |
| Biomarker^{m} | X | X | X | X | X | X | X | X |
| Immunogenicity (fusion | | X | X | X | | | | X |
| polypeptide ADA)ⁿ | | | | | | | | |
| Assessment of VOC^{o} | X | ←Monitor continuously→ | | | | | | |
| Review patient diary^{p} | X | X | X | X | X | X | X | X |
| Review safety card^{q} | X | X | X | X | X | X | X | X |

| | |
|---|---|
| Concomitant medications | ←Monitor continuously (after ICF is signed at Screening)→ |
| Adverse events^{r} | ←Monitor continuously (after ICF is signed at Screening)→ |

^{a}Signed and dated IEC-approved informed consent must be obtained before any study-specific screening procedures are performed.
^{b}Eligibility is assessed at Screening and Day 1.
^{c}Randomization occurs following confirmation of all eligibility requirements.
^{d}To reduce the risk of meningococcal infection (Neisseria meningitidis), all patients are vaccinated with MCV4 and serogroup B meningococcal vaccinations at least 14 days before dosing, if not already vaccinated within 3 years before the first dose (or per national/local guidelines). Participants who initiate study intervention treatment less than 14 days after receiving a meningococcal vaccine must receive treatment with appropriate prophylactic antibiotics until at least 2 weeks after vaccination. Every effort should be made to start the meningococcal vaccination series at least 14 days prior to randomization.
^{e}Height and BMI only at Screening.
^{f}Non-fasting blood samples is obtained.
^{g}Serum pregnancy test is required for all female participants of childbearing potential (i.e., have achieved menarche) to confirm that a female participant is not pregnant prior to first dosing. A urine pregnancy test can be performed if the Screening visit is within 3 weeks of Day 1 visit. A urine (or serum if required by site policy) local pregnancy test is to be performed at all other required timepoints.
^{h}At Screening, supine and standing (orthostatic) blood pressures is performed.
ⁱPredose triplicate 12-lead ECGs are performed on Day 1 only and is performed 3 times at approximately 2, 1, and 0.5 hours before the start of dosing (If this schedule is not feasible, ECGs performed with at least 20 min apart is acceptable). If the scheduled dosing is delayed, predose ECGs performed within 4 hours of dosing do not need to be repeated. Postdose ECGs are performed within 30 min before PK/PD sample collection.
^{j}PK and PD samples are collected at pre-dose and between 2 and 4 hours post dose.
^{k}PD samples are collected to assess serum total and free properdin and CAP activity.
^{l}Injection site reaction evaluation is conducted on dosing days and if a reaction occurs, the evaluation continues until the reaction is fully resolved.
^{m}Blood samples for biomarkers are collected two times prior to receiving the first dose (collect once during Screening and then again at Day 1 pre-dose, the 2 collections should be at least 2 weeks apart). Collection of blood (for serum, citrated and K2—EDTA, or P100 plasma) and urine.
ⁿIn case of any suspected case of hypersensitivity or anaphylaxis additional samples for the assessment of ADA may be collected at or near the time of the event.
^{o}If a VOC crisis occurs, samples are collected as per unscheduled visit.
^{p}The patient records symptoms of VOC in a paper diary throughout the study. The Investigator or qualified designee reviews the patient diary at every visit.
^{q}The Investigator or qualified designee meets with the participant at each visit to ensure they carry the clinical study participant safety card at all times and to review the potential meningococoal infection risks of the fusion polypeptide described herein, and to address any safety concerns of the participant.
^{r}Collection of adverse events and serious adverse events begins after ICF signing.
Abbreviations: ADA = antidrug antibody; BMI = body mass index; CAP = complement alternative pathway; ECG = electrocardiogram; HiB = Haemophilus influenzae type B; HIV = human immunodeficiency virus; ICF = informed consent form; IEC = Independent Ethics Committee; K2—EDTA = dipotassium ethylenediaminetetraacetic acid; MCV4 = tetravalent meningococcal conjugate vaccine; NA = not applicable; PD = pharmacodynamic(s); PK = pharmacokinetic(s); Q4W = once every 4 weeks; VOC = vaso-occlusive crisis.

TABLE 5
Optional PK/PD Sampling for Intensive PK/PD
for Subset of Patients in Cohort 1 and 2

| | Day 2 | Day 4 | Day 86 | Day 88 | Day 92 |
|---|---|---|---|---|---|
| | (24 hours | (72 hours | (24 hours | (72 hours | (168 hours |
| | post | post | post | post | post |
| | Day 1 | Day 1 | Day 85 | Day 85 | Day 85 |
| Study | dose | dose, ±24 | dose | dose, ±24 | dose, ±24 |
| Day^{a} | (±4 hours) | hours) | (±4 hours) | hours) | hours) |
| PK samples | X | X | X | X | X |
| Blood | X | X | X | X | X |
| sample for | | | | | |
| PD panel^{b} | | | | | |

^{a}A subset of Cohort 1 and 2 patients may undergo a more intensive PK/PD sampling specified in this table. All other assessments are to be performed per Table 3, Table 4, and Table 6.
^{b}PD samples are collected to assess serum total and free properdin and CAP activity.
Abbreviations: CAP = complement alternative pathway; PD = pharmacodynamic(s); PK = pharmacokinetic(s).

TABLE 6
Schedule of Activities-Day 99 Through End of Follow-up Period, Cohorts 1 and 2

| | Study Day | | | | | |
|---|---|---|---|---|---|---|
| | | | | | | Complement |
| | | | | | Day 211/ET/ | Activity |
| | Day 99 | Day 127^{a} | Day 155 | Day 183^{a} | Unscheduled | Follow-up^{b} |
| | Window (Day) | | | | | |
| | ±3 | ±3 | ±3 | ±3 | ±3 | ±14 |
| Physical examination | | | | | X | |
| Abbreviated physical examination | X | | X | | | |
| Vital sign measurements | X | | X | | X | |
| Triplicate ECG | X | | X | | X | |
| Hematology^{c} | X | | X | | X | |
| Clinical chemistry^{c} | X | | X | | X | |
| Coagulation^{c} | X | | X | | X | |
| Urinalysis (including spot urine) | X | | X | | X | |
| Serum/urine pregnancy test | X | | X | | X | |
| PK samples | X | | X | | X | |
| Blood samples for PD panel | X | | X | | X | X |
| (serum total and free properdin, CAP | | | | | | |
| activity) | | | | | | |
| Biomarkers^{d} | X | | X | | X | |
| Immunogenicity (fusion polypeptide ADA)^{e} | | | X | | X | |
| Assessment of VOC^{f} | ←Monitor continuously→ | | | | | |
| Review patient diary^{g} | X | X | X | X | X | X |
| Review safety card^{h} | X | X | X | X | X | X |

| | |
|---|---|
| Concomitant medications | ←Monitor continuously→ |
| Adverse eventsⁱ | ←Monitor continuously→ |

^{a}Visit may be performed remotely (phone or video calls).
^{b}If CAP activity has not returned to a normal range or 80% of the baseline level at the end of the Follow-up Period, patients may have additional Follow-up Visits every 12 weeks until CAP activity has been confirmed to either have returned to a normal range or 80% of the baseline level. This visit may be completed at home.
^{c}Non-fasting blood samples is obtained.
^{d}Collection of blood (for serum, citrated and K2—EDTA, or P100 plasma) and urine.
^{e}In case of any suspected case of hypersensitivity or anaphylaxis additional samples for the assessment of ADA may be collected at or near the time of the event.
^{f}Assessment of VOC must be performed by a healthcare provider.
^{g}The patient records symptoms of VOC in a paper diary throughout the study. The Investigator or qualified designee reviews the patient diary at every visit.
^{h}The Investigator or qualified designee meets with the participant at each visit to ensure they carry the clinical study participant safety card at all times and to review the potential meningococcal infection risks of the fusion polypeptide described herein, and to address any safety concerns on the part of the participant.
ⁱCollection of adverse events and serious adverse events begins after ICF signing
Abbreviations: ADA = antidrug antibody; CAP = complement alternative pathway; ECG = electrocardiogram; ET = early termination; ICF = informed consent form; K2—EDTA = dipotassium ethylenediaminetetraacetic acid; PD = pharmacodynamic(s); PK = pharmacokinetic(s); VOC = vaso-occlusive crisis.

TABLE 7
Schedule of Activities - Screening Through Day 43 (Optional Cohort 3) - Q2W Dosing

| | Screening | | | | |
|---|---|---|---|---|---|
| Study Day | Day −56 to Day −1 | Day 1 | Day 15 | Day 29 | Day 43 |
| Window (Day) | NA | NA | ±3 | ±3 | ±3 |
| Informed consent^{a} | X | | | | |
| Eligibility criteria^{b} | X | X | | | |
| Randomization^{c} | | X | | | |
| Confirmation of or | X | | | | |
| administration of | | | | | |
| meningococcal vaccination^{d} | | | | | |
| Confirmation of or | X | | | | |
| administration of HiB and | | | | | |
| S pneumoniae vaccination | | | | | |
| Medical history and | X | | | | |
| demographics | | | | | |
| Physical examination | X | X | | | |
| Abbreviated physical | | | | X | |
| examination | | | | | |
| Height, weight, and BMI^{e} | X | X | | X | |
| Hematology^{f} | X | X | | X | |
| Clinical chemistry^{f} | X | X | | X | |
| Coagulation^{f} | X | X | | X | |
| Hepatitis B and C screen | X | | | | |
| HIV (types 1 and 2) screen | X | | | | |
| Serum/urine pregnancy test^{g} | X | X | X | X | X |
| Urinalysis | X | X | | X | |
| Vital sign measurements^{h} | X | X | X | X | X |
| Window (Day) | NA | NA | ±3 | ±3 | ±3 |
| Triplicate ECGⁱ | X | X | X | X | |
| Study drug administration Q2W | | X | X | X | X |
| PK samples^{j} | | X | X | X | X |
| Blood sample for PD panel^{j, k} | | X | X | X | X |
| Injection site reaction^{l} | | X | X | X | X |
| Biomarker^{m} | X | X | X | X | X |
| Immunogenicity (fusion | | X | X | X | |
| polypeptide ADA)ⁿ | | | | | |
| Assessment of VOC^{o} | X | ←Monitor continuously→ | | | |
| Review patient diary^{p} | X | X | X | X | X |
| Review safety card^{q} | X | X | X | X | X |

| | |
|---|---|
| Concomitant medications | ←Monitor continuously (after ICF is signed at Screening)→ |
| Adverse events^{r} | ←Monitor continuously (after ICF is signed at Screening)→ |

^{a}Signed and dated IEC-approved informed consent must be obtained before any study-specific screening procedures are performed.
^{b}Eligibility is assessed at Screening and Day 1.
^{c}Randomization occurs following confirmation of all eligibility requirements.
^{d}To reduce the risk of meningococcal infection (Neisseria meningitidis), all patients are vaccinated with MCV4 and serogroup B meningococcal vaccinations at least 14 days before dosing, if not already vaccinated within 3 years before the first dose (or per national/local guidelines). Participants who initiate study intervention treatment less than 14 days after receiving a meningococcal vaccine must receive treatment with appropriate prophylactic antibiotics until at least 2 weeks after vaccination. Every effort should be made to start the meningococcal vaccination series at least 14 days prior to randomization.
^{e}Height and BMI only at Screening.
^{f}Non-fasting blood samples are obtained
^{g}Serum pregnancy test is required for all female participants of childbearing potential (i.e., have achieved menarche) to confirm that a female participant is not pregnant prior to first dosing. A urine pregnancy test can be performed if the Screening Visit is within 3 weeks of Day 1 Visit. A urine (or serum if required by site policy) local pregnancy test is to be performed at all other required timepoints.
^{h}At Screening, supine and standing (orthostatic) blood pressures are performed.
ⁱPredose triplicate 12-lead ECGs are performed on Day 1 only and are performed 3 times at approximately 2, 1, and 0.5 hours before the start of dosing (If this schedule is not feasible, ECGs performed with at least 20 min apart is acceptable). If the scheduled dosing is delayed, predose ECGs performed within 4 hours of dosing do not need to be repeated. Postdose ECGs are performed within 30 min before PK/PD sample collection.
^{j}PK and PD samples are collected at pre-dose and between 2 and 4 hours post dose.
^{k}PD samples are collected to assess serum total and free properdin and CAP activity.
^{l}Injection site reaction evaluation is conducted on dosing days and if a reaction occurs, the evaluation continues until the reaction is fully resolved.
^{m}Blood samples for biomarkers are collected 2 times prior to receiving the first dose (collect once during Screening and then again at Day 1 pre-dose, the 2 collections should be at least 2 weeks apart). Collection of blood (for serum, citrated and K2-EDTA, or P100 plasma) and urine.
ⁿIn case of any suspected case of hypersensitivity or anaphylaxis additional samples for the assessment of ADA may be collected at or near the time of the event.
^{o}If a VOC crisis occurs, samples are collected as per unscheduled visit.
^{p}The patient records symptoms of VOC in a paper diary throughout the study. The Investigator or qualified designee reviews the patient diary at every visit.
^{q}The Investigator or qualified designee meets with the participant at each visit to ensure they carry the clinical study participant safety card at all times and to review the potential meningococcal infection risks of the fusion polypeptide described herein, and to address any safety concerns of the participant.
^{r}Collection of adverse events and serious adverse events begins after ICF signing.
Abbreviations: ADA = antidrug antibody; BMI = body mass index; CAP = complement alternative pathway; ECG = electrocardiogram; HiB = Haemophilus influenzae type B; HIV = human immunodeficiency virus; ICF = informed consent form; IEC = Independent Ethics Committee; K2-EDTA = dipotassium ethylenediaminetetraacetic acid; MCV4 = tetravalent meningococcal conjugate vaccine; NA = not applicable; PD = pharmacodynamic(s); PK = pharmacokinetic(s); Q2W = every 2 weeks; VOC = vaso-occlusive crisis.

TABLE 8
Schedule of Activities-Day 57 Through End of Follow-up Period for Multiple Dose Follow-up (Optional
Cohort 3)

| | Study Day | | | | | |
|---|---|---|---|---|---|---|
| | | | | | | Complement |
| | | | | | Day 169/ET/ | Activity |
| | Day 57 | Day 85^{a} | Day 113 | Day 141^{a} | Unscheduled | Follow-up^{b} |
| | Window (Day) | | | | | |
| | ±3 | ±3 | ±3 | ±3 | ±3 | ±14 |
| Physical examination | | | | | X | |
| Abbreviated physical examination | X | | X | | | |
| Vital sign measurements | X | | X | | X | |
| Triplicate ECG | X | | X | | X | |
| Clinical chemistry^{c} | X | | X | | X | |
| Hematology^{c} | X | | X | | X | |
| Coagulation^{c} | X | | X | | X | |
| Urinalysis | X | | X | | X | |
| Serum/urine pregnancy test | X | | X | | X | |
| PK samples | X | | X | | X | |
| Blood samples for PD panel (serum total and | X | | X | | X | X |
| free properdin, CAP activity) | | | | | | |
| Biomarkers^{d} | X | | X | | X | |
| Immunogenicity (fusion polypeptide ADA)^{e} | | | X | | X | |
| Assessment of VOC^{f, g} | ←Monitor continuously→ | | | | | |
| Review patient diary^{g} | X | X | X | X | X | X |
| Review safety card^{h} | X | X | X | X | X | X |

| | |
|---|---|
| Concomitant medications | ←Monitor continuously→ |
| Adverse eventsⁱ | ←Monitor continuously→ |

^{a}Visit may be performed remotely (phone or video calls).
^{b}If CAP activity has not returned to a normal range or 80% of the baseline level at the end of the Follow-up Period, patients may have additional Follow-up Visits every 12 weeks until CAP activity has been confirmed to either have returned to a normal range or 80% of the baseline level. This visit may be completed at home.
^{c}Non-fasting blood samples is obtained.
^{d}Collection of blood (for serum, citrated and K2—EDTA, or Plasma P100 plasma) and urine.
^{e}In case of any suspected case of hypersensitivity or anaphylaxis additional samples for the assessment of ADA may be collected at or near the time of the event.
^{f}Assessment of VOC must be performed by a healthcare provider.
^{g}The patient records symptoms of VOC in a paper diary throughout the study. The Investigator or qualified designee reviews the patient diary at every visit.
^{h}The Investigator or qualified designee meets with the participant at each visit to ensure they carry the clinical trial participant safety card at all times and to review the potential meningococcal infection risks of the fusion polypeptide described herein, and to address any safety concerns on the part of the participant.
ⁱCollection of adverse events and serious adverse events begins after ICF signing.
Abbreviations: ADA = antidrug antibody; CAP: complement alternative pathway; ECG = electrocardiogram; ET = early termination; ICF = informed consent form; K2—EDTA = dipotassium ethylenediaminetetraacetic acid; PD = pharmacodynamic(s); PK = pharmacokinetic(s); VOC = vaso-occlusive crisis.

### 2. Introduction

### 2.1 Study Rationale

The fusion polypeptide described herein (anti-properdin/anti-serum albumin bispecific VHH antibody) is a novel properdin blocking agent being developed for the treatment of diseases involving dysregulated CAP activity. The fusion polypeptide described herein molecule is bispecific, comprising a VHH antibody domain that binds and blocks properdin connected via a linker to a VHH domain that binds serum albumin, thereby conferring an extended circulatory half-life to the molecule.

The fusion polypeptide described herein binds to properdin with a high affinity to prevent stabilization of the CAP complement component C3 (C3) and complement component C5 (C5) convertases that cleave C3 and C5 into their activation products. Quantitative blockade of properdin has been shown to be safe in human based on experience with the properdin binding antibody (see Section 4.3). The fusion polypeptide described herein is currently being evaluated in an ongoing Phase 1 study in healthy adult participants, and doses and dosing regimens for participants in this study were determined using cumulative safety data and an interim PK/PD analysis from participants enrolled in the ongoing Phase 1 study as well as data from the 6-month GLP toxicology in monkeys.

The purpose of this study in patients with SCD is to evaluate the safety, tolerability, efficacy, PK, PD, and immunogenicity of multiple doses and dosing regimens of the fusion polypeptide described herein administered subcutaneously.

The study includes up to 3 cohorts with multiple SC doses and dosing regimens of the open-label fusion polypeptide described herein in adult patients with SCD. Data from this study are anticipated to help design future studies in patients with SCD and other complement-mediated diseases.

### 2.2 Background

A detailed description of the chemistry, pharmacology, and toxicology data available for the fusion polypeptide described herein is provided in the Investigator's Brochure (IB).

### 2.2.1 Chemistry

The fusion polypeptide described herein is a recombinant, humanized VHH bispecific antibody that binds to human properdin and serum albumin. The antibody consists of a single polypeptide chain of 256 amino acids, which is comprised of an anti-albumin domain at the N-terminus that is fused to a C-terminal anti-properdin domain via a 15 amino acid linker. The variable region domains that form the serum albumin and properdin binding sites consist of llama complementarity determining regions grafted into human germline frameworks. Within the framework regions, llama residues at 11 positions were left unchanged to maintain antigen binding, aqueous solubility, and overall stability. There are 2 intrachain disulfide bonds, 1 disulfide bond localized in each VHH domain. The theoretical average molecular mass of the antibody is 27,350.2 Da. At pH 7.4, the fusion polypeptide described herein exhibited a binding dissociation constant (K_{D}) of 323 pM for human properdin and K_{D} of 439 pM for human serum albumin. The IC₅₀ for blockade of human CAP hemolysis by the fusion polypeptide described herein (20% v/v final serum) was 29 nM. The fusion polypeptide described herein blocked C3 fragment, properdin and complement component C9 (C9) deposition onto a myeloperoxidase substrate by human serum (20% v/v) with IC₅₀ values of approximately 20 nM (C3), 15 nM (properdin) and 19 nM (C9).

### 2.2.2 Nonclinical Pharmacology

In a study of the potency of blockade of in vitro CAP hemolysis by the fusion polypeptide described herein using sera across a range of species, significant species cross reactivity was observed only when using sera from cynomolgus and rhesus macaque. CAP hemolytic activities in the sera of mouse, rat, guinea pig, minipig, beagle and rabbit were not inhibited by the highest concentration of the fusion polypeptide described herein examined (>100 μg/mL). The IC₅₀ for blockade of cynomolgus CAP hemolysis by the fusion polypeptide described herein (20% v/v final serum) was 47 nM. The fusion polypeptide described herein exhibited a binding K_{D} of 2.9 nM for cynomolgus properdin and K_{D} of 2.1 nM for cynomolgus serum albumin. The fusion polypeptide described herein blocked C3 fragment, properdin and C9 deposition onto a myeloperoxidase substrate by cynomolgus monkey serum (20% v/v) with IC₅₀ values of approximately 11 nM (C3), 9 nM (properdin) and 17 nM (C9). The lack of species cross reactivity beyond primates described above prevented testing the biologic activity of the fusion polypeptide described herein in traditional rodent models of diseases involving dysregulated complement activity. Collectively therefore, the nonclinical in vitro and in vivo studies to assess the pharmacologic, PK, PD and toxicologic properties of the fusion polypeptide described herein are being performed in cynomolgus monkeys.

### 2.2.3 Toxicology

Nonclinical safety profile of the fusion polypeptide described herein has been evaluated in an in vitro GLP tissue cross reactivity (TCR) study and in both a non-GLP and GLP in vivo studies in cynomolgus monkeys. In GLP toxicology studies the fusion polypeptide described herein was administered by intravenous (IV) administration (up to single dose of 100 mg/kg) and SC administration (up to 26 weekly doses of 300 mg/kg/week). The fusion polypeptide described herein did not demonstrate any non-specific binding to human tissues in the TCR study. Based on the absence of any adverse systemic or local toxicity in cynomolgus monkeys, 300 mg/kg/week was considered the no observed adverse effect level (NOAEL) for SC administration and 100 mg/kg was considered as the NOAEL for IV administration of the fusion polypeptide described herein. Systemic exposures (maximum observed serum concentration [Cₘₐₓ] and area under the concentration-time curve from time 0 to 168 hours [AUC₀₋₁₆₈] of 8,570 μg/mL and 1,160,000 μg·hr/mL, respectively), after the last dose at the NOAEL in the SC group, yielded exposure multiples of ~30-fold to the projected exposures at the anticipated human dose of 300 mg QW. Antidrug antibodies, when observed in a very small number of monkeys, did not have any impact on systemic exposure or toxicity profile.

### 2.3 Benefit/Risk Assessment

There may be potential benefits to patients with SCD (See Section 2.3.2). Identified and potential risks are described below. More detailed information about the known and expected benefits and risks and reasonably expected AEs of the fusion polypeptide described herein may be found in the IB. Potential risk mitigation strategies are described in Table 9.

### 2.3.1 Risk Assessment

Besides the first-in-human (FIH) Phase 1 study in healthy participants, this study is the second human exposure to the fusion polypeptide described herein and the first exposure to patients with SCD. As there is limited clinical experience to date, the potential risks are based on the class of the molecule and its mechanism of action. No potential risks were identified from the repeat dose toxicity study in cynomolgus monkeys with the fusion polypeptide described herein after 26 weekly SC doses up to 300 mg/kg/week or after a single IV dose up to 100 mg/kg and no safety concerns in healthy participants have been identified as of 11 Mar. 2022 after 5 weekly SC doses up to 150 mg or after a single SC dose of 1200 mg.

2.3.1.1 Neisseria meningitidis Infections

Increased susceptibility to infection with Neisseria meningitidis is a known risk associated with properdin deficiency and has been well described with properdin-deficient patients (Figueroa, 1991). Similar to properdin deficiency, the main risk associated with the use of the fusion polypeptide described herein (properdin inhibitor) is expected to be the risk of meningococcal infections. Specific risk mitigation measures are in place to address this risk.

Clinically, the risk of N. meningitidis is mitigated in patients with properdin deficiency by vaccinating all patients against N. meningitidis with tetravalent meningococcal conjugate vaccine (MCV4) and serogroup B vaccines before dosing.

Patients are vaccinated with MCV4 and serogroup B meningococcal vaccinations at least 14 days before dosing, if not already vaccinated within 3 years before the first dose (or per national/local guidelines). Participants who initiate study intervention treatment less than 14 days after receiving a meningococcal vaccine must receive treatment with appropriate prophylactic antibiotics until at least 2 weeks after vaccination. Every effort should be made to start the meningococcal vaccination series at least 14 days prior to randomization.

Additionally, participants may be treated with prophylactic antibiotics at the Investigator's discretion.

### 2.3.1.2 Immunogenicity and Hypersensitivity

The fusion polypeptide described herein has the potential to be immunogenic and may be associated with hypersensitivity reactions. Some healthy participants are also known to have pre-existing antibody to VHH antibodies. Antibodies to the fusion polypeptides described herein have been observed in 14 of 100 healthy participant serum samples tested in an in vitro screening assay.

Monitoring of immunogenicity for the fusion polypeptides described herein in place for this study as specified in the SoA (Section 1.3).

TABLE 9
Potential Risks and Mitigation Strategies

| | | |
|---|---|---|
| Potential Risk of | Summary of Data/Rationale | |
| Clinical Significance | for Risk | Mitigation Strategy |

Treatment with the fusion polypeptide described herein

| | | |
|---|---|---|
| Meningococcal | The fusion polypeptide blocks | Participants must be vaccinated |
| infection | properdin which can result in | against all available serotypes of |
| | increased susceptibility to | N. meningitidis (A, C, Y, W 135, and |
| | Neisseria meningitidis, likely with | B). However, vaccination may not be |
| | fulminant clinical manifestations. | sufficient to prevent meningococcal |
| | | infection. |
| | | Prophylactic antibiotics are used at |
| | | the investigator's discretion. Timing |
| | | for meningococcal vaccinations can |
| | | be mandated per local/national |
| | | guidelines. |
| | | (See Sections 1.3 and 2.3.1.1) |
| | | Participants are provided a safety |
| | | card to carry at all times and for 5 |
| | | months after the final dose of the |
| | | fusion polypeptide described herein. |
| Immunogenicity | Treatment with any therapeutic | Algorithm for hypersensitivity reaction |
| | protein may induce an immune | management; Stopping rules and |
| | response. Occasionally, this | staggered dosing |
| | immune response is clinically | |
| | meaningful. The consequences of | |
| | an immune reaction to a | |
| | therapeutic protein range from | |
| | transient appearance of | |
| | antibodies, without any clinical | |
| | consequence, to severe, life- | |
| | threatening conditions. Potential | |
| | clinical consequences also may | |
| | include severe | |
| | hypersensitivity-type reactions, | |
| | decrease in efficacy and induction | |
| | of autoimmunity. | |

### 2.3.1.3 Coronavirus Disease 2019

The SARS-COV-2 disease (coronavirus disease 2019 [COVID-19]) global pandemic is active in many countries at the time of this protocol amendment. Given this unique circumstance, specific consideration has been given to the risks and benefits of the study as they may be related to COVID-19, and the global and local changes that exist as a result of the pandemic.

### 2.3.2. Benefit Assessment

The potential benefit of the fusion polypeptide described herein treatment for SCD is being measured by assessments of anemia and hemolysis. An increase in hemoglobin by 1 g/dL from baseline is considered clinically meaningful. In addition, a positive treatment effect would also be shown by a decrease in hemolysis, including improvements in the serum levels of markers of hemolysis (i.e., lactate dehydrogenase [LDH], indirect bilirubin, and haptoglobin). Other exploratory endpoints (vaso-occlusive crisis [VOC], etc.) may be assessed.

### 2.3.2 Overall Benefit: Risk Conclusion

The fusion polypeptide described herein has been and is being evaluated in an ongoing Phase 1 study. The study described herein is the second human exposure to the fusion polypeptide described herein.

The present study is conducted in patients with SCD, and dosing is initiated based on review of safety, tolerability, and PK/PD data from the Phase 1 study. The doses administered in the current study are expected to produce exposure lower than the highest exposure tested in the previous study, and the expected exposure in patients is lower than the NOAEL exposure established in 6-week and 6-month GLP monkey toxicology studies. Strict inclusion/exclusion criteria, with a robust safety monitoring and risk mitigation plan are in place. A DMC evaluates the available study data at prespecified time points for participant safety and make recommendations on dose modification or termination of the study. The selected doses are intended to deliver complete inhibition of properdin, providing potential benefit of the fusion polypeptide described herein to patients with SCD, with a positive benefit/risk ratio.

The data obtained from this study is expected to inform future clinical studies in patients with SCD.

### 3. Objectives and Endpoints

The study objectives and corresponding endpoints are presented in Table 10.

TABLE 10
Mapping Objectives to Endpoints for Patients with Sickle Cell Disease

| Objectives | Endpoints |
|---|---|
| Primary | |
| To assess the safety and tolerability of the SC | Safety assessed by incidence of TEAEs and |
| administration of the fusion polypeptides | SAEs, physical examination, vital sign |
| described herein in patients with SCD | measurements, clinical laboratory, and |
| | electrocardiogram results through Day 211 |
| | (Cohorts 1 and 2) and through Day 169 (Optional |
| | Cohort 3) |
| Secondary | |
| To assess the multiple-dose PK of the SC | Serum multiple-dose PK profiles of the fusion |
| administration of the fusion polypeptide | polypeptide described herein through Day 211 |
| described herein | (Cohorts 1 and 2) and through Day 169 (Optional |
| | Cohort 3) |
| To assess the PD effects of the SC | Change in serum concentrations of total and free |
| administration of fusion polypeptide | properdin over time through Day 211 (Cohorts 1 |
| described herein | and 2) and through Day 169 (Optional Cohort 3) |
| | Change in CAP activity using the Wieslab AP |
| | assay through Day 211 (Cohorts 1 and 2) and |
| | through Day 169 (Optional Cohort 3) |
| To assess the effect of the fusion polypeptides | Change from baseline in complement biomarkers |
| described herein on complement biomarkers | through Week 12 (Cohorts 1 and 2) |
| To assess the effect of the fusion polypeptide | Change from baseline in hemoglobin levels at |
| described herein on hemolysis | Week 12 (Cohorts 1 and 2) |
| | Change from baseline in hemolysis markers (e.g., |
| | lactate dehydrogenase, reticulocytes, and |
| | bilirubin) and hemopexin at Week 12 (Cohorts 1 |
| | and 2) |
| To assess the immunogenicity of the SC | Incidence of ADAs to the fusion polypeptide |
| administration of the fusion polypeptide | described herein through Day 211 (Cohorts 1 and |
| described herein | 2) and through Day 169 for Optional Cohort 3 |
| Exploratory | |
| To explore the effect of the fusion polypeptide | Change from baseline in VOC related biomarkers |
| described herein on vaso-occlusion markers | through Week 12 (Cohorts 1 and 2) |
| To explore the clinical efficacy of the SC of | VOC events (rate of VOC, time to first VOC) |
| the fusion polypeptide described herein in SCD | through Week 12 (Cohorts 1 and 2) |

Abbreviations: ADA = antidrug antibody; AP = alternative pathway; CAP = complement alternative pathway; PD = pharmacodynamic(s); PK = pharmacokinetic(s); SAE = serious adverse event; SC = subcutaneous; SCD = sickle cell disease; TEAE = treatment-emergent adverse event; VOC = vaso-occlusive crisis.

### 4. Study Design

### 4.1 Overall Design

This is a Phase 2a study, with up to 3 multiple dose cohorts of open-label SC of the fusion polypeptide described herein in adult patients with SCD (HbSS and HbSβ⁰-thalassemia).

The study is conducted in up to 30 adult patients with SCD enrolled in up to 3 open-label cohorts (Cohorts 1, 2 and 3 [optional]) to receive multiple SC doses of the open-label fusion polypeptide described herein. The fusion polypeptide described herein is administered as described in Table 11.

TABLE 11
The Fusion Polypeptide Dosing Cohorts

| | | | | | Number |
|---|---|---|---|---|---|
| | | | Route of | | of Doses/ |
| | | | Adminis- | Planned | Dose |
| Cohort | N | Study Drug | tration | Dose | Interval |
| 1 | Up to 12 | The fusion | SC | 300 mg | QW × 13 |
| | maximum of 6 | polypeptide | | | |
| | on stable dose | described | | | |
| | of hydroxyurea | herein | | | |
| 2 | Up to 12 | The fusion | SC | 600 mg | Q4W × 4 |
| | maximum of 6 | polypeptide | | | |
| | on stable dose | described | | | |
| | of hydroxyurea | herein | | | |
| 3 (op- | Up to 6 | The fusion | SC | 300 mg | Q2W × 4 |
| tional) | maximum of 3 | polypeptide | | | |
| | on stable dose | described | | | |
| | of hydroxyurea | herein | | | |

Abbreviations: N = number of participants; QW = once weekly; Q2W = once every 2 weeks; Q4W = once every 4 weeks; SC = subcutaneous.

The dose and dosing intervals for Cohorts 1 and 2 were determined using cumulative safety data, an interim PK/PD analysis from participants enrolled in the Phase 1 study, and data from the 6-month GLP toxicology study in monkeys. Optional Cohort 3 is initiated after evaluation of safety and PK/PD data from Cohorts 1 and 2.

Cohort 1 and Cohort 2 run in parallel, and patients are randomized 1:1 to either of the cohorts on determination of eligibility. The decision to initiate Optional Cohort 3 is made at the discretion of the Investigator and is based on analysis of PK/PD and safety after at least 8 patients from Cohorts 1 and 2 (4 from each cohort) have enrolled. Furthermore, enrollment of Optional Cohort 3 starts after Cohorts 1 and 2 are fully enrolled. Each cohort is stratified to ensure patients with SCD who are treated with a stable dose of hydroxyurea and patients with SCD who are not currently treated with hydroxyurea are included. For patients who were previously treated with hydroxyurea but are not currently on hydroxyurea, treatment must be stopped at least 30 days prior to providing informed consent.

This study uses an independent DMC to monitor safety and to perform the planned interim analyses of the study. At the discretion of the Investigator, and after consultation with the DMC, additional participants with SCD may be enrolled as replacement participants if a participant discontinues during the Dosing Period for reasons other than drug related AEs.

### 4.2 Scientific Rationale for Study Design

The initial indication for the fusion polypeptide described herein is SCD. SCD affects about 20 to 25 million people worldwide (Aliyu, 2008) and in the US, approximately 100,000 people are affected (Hassell, 2010). The prevalence of SCD newborns and SCD carriers in the EU is approximately 1 to 5 in 10,000 and 1 in 150, respectively (Engert, 2016). The few available life expectancy estimates for SCD patients in the US or EU vary widely from 45 to 65 years which is approximately 20 years lower than the general population (Gardner, 2016; Lubeck, 2019; Payne, 2020; Platt, 1994).

SCD is a group of inherited disorders. A mutation in the β-hemoglobin gene is responsible for the synthesis of sickle hemoglobin (HbS). The most common genotypes in SCD are HbSS, HbSC, and HbSβ⁺ thalassemia. The most common clinical manifestations of SCD are chronic hemolysis and VOC (Kato, 2018; Pecker, 2021).

HbS has abnormal physicochemical properties and is prone to polymerization under low oxygen concentration causing deformation of red blood cells (RBCs) with the characteristic sickle shape. Sickling has numerous adverse outcomes on RBCs and on multiple organs. Sickled RBCs have limited life span due to hemolysis. Hemolysis is thought to occur principally through extravascular phagocytosis (approximately ⅔) and intravascular (approximately ⅓) hemolysis, which leads to anemia. Intravascular hemolysis of sickle RBCs leads to release of free hemoglobin, which in turn activates CAP (via free heme) and depletes nitric oxide, contributing to endothelial damage. Hemolysis and sickling of the RBCs leads to endothelial cell activation with increased adhesion molecule expression and activation of neutrophils, monocytes, and platelets (Kato, 2018). VOC, as a result of these processes, leads to obstruction of blood flow to vital organs such as the kidneys, liver, lungs, and heart, promoting ischemia, acute episodes of pain, and necrosis. The ensuing ischemic/reperfusion injury leads to the generation of reactive oxygen species. This in turn leads to a chronic inflammatory state (Kato, 2018; Piel, 2017). Patients also have increased vulnerability to infections, particularly from encapsulated bacteria, as a result of functional or actual asplenia. Together, these mechanisms contribute to the development of chronic organ damage including sickle nephropathy, pulmonary hypertension, avascular necrosis of the bone, chronic lung disease, and shortened life expectancy (Kato, 2018).

Universal newborn screening is established in the US to enable early diagnosis and treatment of infants with SCD to reduce morbidity and mortality. It is recommended that all infants with HbSS and HbSβ⁰-thalassemia receive penicillin prophylaxis and 23-valent-pneumococcal polysaccharide vaccine to prevent invasive pneumococcal disease (Kato, 2018; Pecker, 2021).

Hydroxyurea, RBC transfusion, and opioids are the treatments commonly used to manage the symptoms of SCD. There are several approved novel drugs for the treatment of sickle cell complications in recent years: L-glutamine (Endari®), voxelotor (Oxbryta®), and crizanlizumab (Adakveo®). However, none of these treatments addresses both anemia (hemolysis) and VOC. The only curative treatment option for SCD is hematopoietic stem cell transplantation. However, this is reserved for severe patients due to the risk of life-threatening complications (Pecker, 2021).

Recently, much attention has been given to the role of the innate immune system in SCD, and in particular the role of complement activation in the pathophysiology of SCD (Tampaki, 2021; Varelas, 2021). In Investigator-initiated studies, eculizumab has demonstrated clinical effect in SCD patients with delayed hemolytic transfusion reaction, VOC, and drug-induced immune hemolytic anemia (Chonat, 2020). Compared to C5 inhibitors, CAP inhibitors have potential advantages in treatment of SCD. An increasing number of publications support the hypothesis that sickle RBCs are the focal point of CAP activation, which triggers C3 opsonization on the cell surface as well as complement-mediated RBC hemolysis. Intravascular hemolysis is one of the main causes of anemia and also contributes to further amplification of CAP activation by releasing free heme from RBCs. C3 opsonization of sickle RBCs also promotes anemia through extravascular hemolysis via the reticuloendothelial system. Furthermore, C3 opsonization has been shown to be a key contributor to VOC. It has also been demonstrated that C3 opsonization can be precipitated by exposure of phosphatidyl serine on sickle RBCs and contributes to VOC by enhancing its interaction with adhesion molecules such as P-selectin and complement receptor 3 on activated endothelial cells (Lombardi, 2019). Thus, SCD nonclinical literature collectively underscores the role of CAP activation in SCD pathophysiology.

The fusion polypeptide described herein binds with high affinity to human properdin, which is a component of the CAP, preventing it from stabilizing the CAP C3 and C5 convertases that cleave C3 and C5 into their activation products. By binding properdin, the fusion polypeptide described herein prevents activation of the alternative complement system and thereby has the potential to treat SCD. In support of this, studies carried out in a mouse model of SCD demonstrated that pretreatment of animals with a mouse anti-properdin antibody significantly ameliorated signs of hemolysis and vaso-occlusion, which are 2 main clinical features of SCD.

The current nonclinical data and data from the first in human study support further investigation of the fusion polypeptide described herein as a potential for treatment of patients with SCD. This study is designed to allow preliminary evaluation of changes in SCD disease-related biomarkers and to guide the design of further clinical studies in patients with SCD.

### 4.3 Justification for Dose

The dose and the frequency of dosing is based on all available data including the overall safety, tolerability, PK/PD modeling from the ongoing cohorts in the Phase 1 study; available nonclinical data including PK, PD, and efficacy in SCD mouse models, and toxicology data from the GLP 6-week and 6-month studies in cynomolgus monkeys.

A preliminary PK/PD model has been established based on data from the Phase 1 study in healthy participants. This semi-mechanistic model assumed monovalent fusion polypeptides described herein binding to trimeric properdin with 3 binding sites. The relationship between free properdin and CAP activity was characterized by a sigmoidal Emax model. The model provided good fits to the observed data (the fusion polypeptide described herein, total and free properdin, and CAP activity) judged by the model diagnostics. To estimate the therapeutic dose in SCD patients, the following assumptions have been made:
Complete suppression of CAP activity (<1% of baseline activity) is needed for clinical efficacy based on data from mouse SCD model (described in detail in the fusion polypeptide described herein IB)
The baseline properdin concentration is about 20% higher in sickle cell patients than in healthy subjects (Strauss, 1977)
   In addition, the fusion polypeptide described herein clearance in sickle cell patients was set as similar to or 40% higher (seen in other antibody treatments for SCD (Crizanlizumab, 2019)) than that of the healthy participants to evaluate the impact of increased clearance on exposure and CAP inhibition. Based on these assumptions and analysis, it is predicted that a dose of 300 mg QW could inhibit the CAP activity to <1% of the baseline values (upper limit of the 90% prediction interval) and maintain this effect during the Treatment Period. A dose of 600 mg Q4W or 300 mg Q2W was also shown to inhibit the CAP activity <1% during majority of the dosing interval. The CAP activity slowly recovers and returns to baseline after terminating dosing of the fusion polypeptide described herein. The safety margin at 300 mg QW is approximately 30-fold based on the NOAEL exposure established in the 6-month (or 26-week) monkey toxicology study. The safety margin at 600 mg Q4W or 300 mg Q2W is approximately 60-fold, based on the NOAEL exposure.

### 4.4 End of Study Definition

A participant is considered to have completed the study if he/she has completed all phases of the study including the last scheduled procedure shown in the SoA (Section 1.3).

The end of the study (EOS) is defined as the date the last participant completes the last visit as shown in the SoA (Section 1.3).

### 5. Study Population

Prospective approval of protocol deviations to recruitment and enrollment criteria, also known as protocol waivers or exemptions, is not permitted.

### 5.1 Inclusion Criteria

### Age

1. Participant must be 18 to 65 years of age inclusive, at the time of signing the informed consent.

### Type of Participant and Disease Characteristics

2. Confirmed diagnosis of SCD (HbSS, or HbSβ⁰-thalassemia).

### Weight

3. Body weight ≥40 kg (inclusive) at Screening.

### Sex

4. Contraceptive use by men or women should be consistent with local regulations regarding the methods of contraception for those participating in clinical studies. Female participants of childbearing potential and male participants with female partners of childbearing potential must be willing to follow protocol-specified contraception guidance while on treatment and for at least 6 months after last dose of study drug.

### Other Inclusion Criteria

5. Hemoglobin between 5.5 and 10 g/dL at Screening.
6. Have had 1 to 10 VOCs in the past 12 months.
7. Patients receiving hydroxyurea must have been on a stable dose for ≥3 months prior to providing informed consent, with no anticipated need for dose adjustment during the study. For patients who previously used hydroxyurea but are not currently on hydroxyurea treatment (due to non-responsiveness, intolerance, or unwillingness to take hydroxyurea), hydroxyurea treatment must have been discontinued at least 30 days prior to providing informed consent.
8. Patients are vaccinated with MCV4 and serogroup B meningococcal vaccinations at least 14 days before dosing, if not already vaccinated within 3 years before the first dose (or per national/local guidelines). Participants who initiate study intervention treatment less than 14 days after receiving a meningococcal vaccine must receive treatment with appropriate prophylactic antibiotics until at least 2 weeks after vaccination.
9. Haemophilus influenzae type b (Hib) and Streptococcus pneumoniae vaccination are up to date according to current national/local vaccination guidelines for patients with SCD.
10. Must be willing to abide by all study requirements and restrictions.

### Informed Consent

11. Capable of giving signed informed consent (or assent, as applicable), which includes compliance with the requirements and restrictions listed in the ICF and in this protocol.

### 5.2 Exclusion Criteria

Participants are excluded from the study if any of the following criteria apply:
1. Planned initiation, termination, or dose alteration of hydroxyurea during the study.
2. Receiving Voxelotor (OXBRYTA) or crizanlizumab (ADAKVEO) within 60 days of providing informed consent.
3. Receiving treatment with recombinant human erythropoetins (e.g., epoetin alfa).
4. Treated with complement inhibitors within 6 months prior to the first dose.
5. Patients who are on chronic transfusion or receive a transfusion within 60 days of first dose.
6. Any significant disease or disorder which, in the opinion of the Investigator, may put the participant at risk.
7. History of complement deficiency.
8. History of N. meningitidis, S. pneumoniae, or H. influenzae infection.
9. History of malignancy with the exception of a nonmelanoma skin cancer or carcinoma in situ of the cervix that has been treated with no evidence of recurrence within 5 years.
10. Evidence of hepatitis B (positive hepatitis surface antigen [HBsAg] or positive core antibody (anti-HBc) with negative surface antibody [anti-HBs]) or hepatitis C viral infection (hepatitis C virus [HCV] antibody positive, except for patients with documented successful treatment and documented sustained virologic response) at Screening.
11. Active systemic bacterial, viral, or fungal infection within 14 days prior to dosing.

### Prior/Concurrent Clinical Study Experience

12. Participation (i.e., last protocol-required study visit) in a clinical study within 90 days or 5 half-lives of the investigational agent, whichever is longer, before initiation of dosing on Day 1.
13. Participation in more than 1 clinical study of a monoclonal antibody (mAb), or participation (i.e., last protocol required study visit) in a clinical study of a mAb within the 6 months or 5 half-lives of the mAb, whichever is longer, prior to Screening, during which the participant was exposed to the active study drug.

### Diagnostic Assessments

14. Severe renal impairment (estimated glomerular filtration rate [eGFR]<30 mL/min/1.73 m²) or on chronic dialysis.

### Other Exclusions

15. Female participants who are pregnant or breastfeeding.
16. History of allergy or hypersensitivity to excipients of the fusion polypeptide described herein (e.g., polysorbate 80).

### 5.3 Lifestyle Considerations

Not applicable, the present study described herein does not include any specific lifestyle considerations.

### 5.4 Screen Failures

Screen failures are defined as participants who consent to participate in the clinical study but are not subsequently assigned to study drug due to failure to meet eligibility criteria. A minimal set of screen failure information is required to ensure transparent reporting of screen failure participants to meet the Consolidated Standards of Reporting Trials publishing requirements and to respond to queries from regulatory authorities. Minimal information includes demography, screen failure details (e.g., failed eligibility criteria), and any AEs, including any SAEs and any related concomitant medication, occurring during the Screening Period.

Participants who do not meet the criteria for participation in this study (i.e., screen failures) due to a reason that is expected to resolve or has resolved may be rescreened based on discussion and agreement between the Investigator and the Medical Monitor. Any abnormal laboratory parameter(s) results outside of the reference range at Screening may be repeated per the Investigator's discretion for the purpose of further determining eligibility.

### 6. Study Intervention

Study intervention is defined as any investigational intervention(s), marketed product(s), placebo, or medical device(s) intended to be administered to a study participant according to the study protocol. For the present study described herein, the study intervention is the fusion polypeptide described herein and also referred to as study drug throughout this study protocol.

### 6.1 Study Intervention Administered

The study drug composition of the fusion polypeptide described herein and doses to be administered (open-label, SC) in this study are presented in Table 12.

TABLE 12
Dose Chart for Study

| Characteristics | Subcutaneous Fusion Polypeptides |
|---|---|
| Dosage | The fusion polypeptide described herein is formulated |
| formulation | at pH 5.4 and each vial contains 300 mg of the fusion |
| | polypeptide described herein in 20 mM sodium acetate, |
| | 250 mM sucrose, and 0.05% polysorbate-80. |
| | The concentration is 150 mg/mL. |
| Unit dose | 300 mg, 600 mg |
| strength(s)/ | |
| dosage level(s) | |
| Route of | SC |
| administration | |
| Dosing | The SC fusion polypeptide described herein is |
| instructions | administered as a single manual push for the 300 mg |
| | dose and as 2 manual pushes for the 600 mg dose. |
| Packaging and | Each vial is packaged into a kit. There is 1 vial per |
| labeling | kit. Both vials and kits are labeled according to the |
| | protocol and local regulatory requirements. |

Abbreviation: SC = subcutaneous.

In the event of missed doses, unscheduled dosing may be required to ensure therapeutic coverage. The unscheduled dose is determined by the study Clinical Pharmacologist on an individual basis.

### 6.2 Preparation/Handling/Storage/Accountability

Details regarding preparation, handling, storage, accountability, and administration of the study drug are discussed below. Additional guidance is provided in the pharmacy manual.
1. The fusion polypeptide described herein is to be stored at 2-8 °C. The Investigator or designee must confirm appropriate temperature conditions have been maintained during transit for all study drug received and any discrepancies are reported and resolved before use of the study drug.
2. Only participants enrolled in the study may receive study drug and only authorized site staff may supply or administer study drug. All study drugs must be stored in a secure, environmentally controlled, and monitored (manual or automated) area in accordance with the labeled storage conditions with access limited to the Investigator and authorized site staff.
3. The pharmacy staff at the Investigator's site are responsible for study drug accountability, reconciliation, and record maintenance (i.e., receipt, reconciliation, and final disposition records). The site's pharmacy assembles the supplies into individual, labelled participant dose syringes and perform Qualified Person certification of the assembled product. Further guidance regarding preparation, handling, storage, and accountability and information for the final disposition of unused study intervention is provided in the Pharmacy Manual.

### 6.3 Measures to Minimize Bias: Randomization and Blinding

All eligible participants who meet all inclusion and no exclusion criteria in Cohorts 1, 2, and optional Cohort 3 receive the open-label fusion polypeptide described herein.

### 6.4 Study Intervention Compliance

The administration of study intervention to participants are under the supervision of the Investigator or their designee to ensure that participants receive the appropriate dose at the appropriate time points during the study.

The date and time of each dose administered in the clinic is recorded in the source documents and case report form (CRF).

The dose of study intervention and study participant identification is confirmed at the time of dosing by a member of the study site staff other than the person administering the study intervention.

For additional information on study intervention compliance and management, refer to the Pharmacy Manual.
6.5 Concomitant Therapy

Any medication (including over-the-counter or prescription medicines, vitamins, and/or herbal supplements), vaccine, or other specific categories of interest that the participant is receiving at the time of enrollment or receives during the study must be recorded along with:
Reason for use
Dates of administration including start and end dates
Dosage information including dose and frequency

The Medical Monitor should be contacted if there are any questions regarding concomitant or prior therapy.

### 6.5.1 Allowed Medicine and Therapy

Multivitamins, contraceptives, and paracetamol (i.e., acetaminophen, at doses of □ 2 g/day) are permitted for use during the study at the Investigator's discretion. Topical skin products should not be administered at the site of study drug injection from 24 hours prior until 24 hours following study drug administration.

See Section 8.2.8 for details on administration and duration of treatment with prophylactic antibiotics as concomitant medication to mitigate the risk of N. meningitidis infection associated with complement inhibition.

Other concomitant medication may be considered on a case-by-case basis by the Investigator in consultation with the Medical Monitor if required. Concomitant procedures are not allowed unless medically indicated.

Medication and therapy for SCD are allowed except for those presented in Section 6.5.2.

### 6.5.2 Disallowed Medicine and Therapy

During the Screening and Treatment Period, if patients are not currently treated with hydroxyurea, hydroxyurea should not be initiated. If patients are on stable dose of hydroxyurea, the dose of hydroxyurea should not be altered or terminated. The use of voxelotor, crizanlizumab, erythropoetins, other complement inhibitors, and transfusion are not allowed. If any of the above medication/therapy is used, the patient is discontinued as per Section 7.1. The above medications/therapy are allowed during the safety Follow-Up period.

### 6.6 Dose Modification

Decisions to continue or modify dosing are made by the Investigator and/or DMC after review of the safety data. The DMC may also make recommendations regarding safety issues, study conduct, or study suspension.

6.7 Intervention after the End of the Study

No follow-up intervention is planned.

### 7. Discontinuation of Study Intervention and Participant Discontinuation/Withdrawal

### 7.1 Discontinuation of Study Intervention

In rare instances, it may be necessary for a participant to permanently discontinue (definitive discontinuation) the study intervention. If the study intervention is definitively discontinued, the participant should remain in the study to be evaluated for safety follow-up. See the SoA for data to be collected at the time of discontinuation of study intervention and follow-up and for any further evaluations that need to be completed (Section 1.3).

### 7.2 Stopping Criteria

### 7.2.1 Individual Stopping Rules

Participants should be considered for discontinuation from intervention if any of the following occur during the study:
Serious hypersensitivity reaction;
Grade 3 or higher injection site reactions;
Severe uncontrolled infection;
Serious N. meningitidis, S. pneumoniae, or H. influenzae infections;
Use of disallowed medication as defined in Section 6.5.2;
Pregnancy or planned pregnancy; or
Any AE, laboratory abnormality, or intercurrent illness which, in the judgment of the Investigator, presents a substantial clinical risk to the patient with continued study drug dosing.

### 7.2.2 Study Stopping Rules

The study may be terminated at the recommendation of the DMC, if the following occur and are deemed to be related to the study drug:
Two or more meningococcal infections;
Two or more serious (Grade 3) pneumococcal infections;
Two or more serious (Grade 3) H. influenzae infections;
One meningococcal, pneumococcal, or H. influenzae infection resulting in a fatal outcome.

### 7.3 Participant Discontinuation/Withdrawal From the Study

All efforts should be made to ensure participants are willing to comply with study participation prior to conducting the screening procedures. The study staff should notify the Investigator and their site monitor of all study withdrawals as soon as possible. The reason for participant discontinuation must be recorded in the source documents and electronic case report form (eCRF).

A participant may withdraw from the study at any time at his/her own request, or may be withdrawn at any time at the discretion of the Investigator for safety, behavioral, compliance, or administrative reasons. This is expected to be uncommon.

At the time of discontinuing from the study, if possible, an Early Discontinuation Visit should be conducted, as shown in the SoA (Section 1.3). Refer to the SoA for data to be collected at the time of study discontinuation and follow-up and for any further evaluations that need to be completed.

The participant is permanently discontinued both from the study drug and from the study at that time.

If the participant withdraws consent for disclosure of future information, the Investigator may retain and continue to use any data collected before such a withdrawal of consent.

If a participant withdraws from the study, he/she may request destruction of any samples taken and not tested, and the Investigator must document this in the site study records.

Participants who discontinue during the Screening or Dosing Period for reasons other than drug-related AEs may be replaced.

### 7.4 Lost to Follow-Up

A participant is considered lost to follow-up if he or she repeatedly fails to return for scheduled visits and is unable to be contacted by the study site.

The following actions must be taken if a participant fails to return to the clinic for a required study visit:
The site must attempt to contact the participant and reschedule the missed visit as soon as possible and counsel the participant on the importance of maintaining the assigned visit schedule and ascertain whether or not the participant wishes to and/or should continue in the study.
Before a participant is deemed lost to follow-up, the Investigator or designee must make every effort to regain contact with the participant (where possible, email, 3 telephone calls and, if necessary, a certified letter to the participant's last known mailing address or local equivalent methods). These contact attempts should be documented in the participant's medical record.
Should the participant continue to be unreachable, he/she is considered to lost to follow up.

### 8. Study Assessments and Procedures

Study procedures and their timing are summarized in the SoA (Section 1.3). Protocol waivers or exemptions are not allowed.

Immediate safety concerns should be discussed with the Investigator immediately upon occurrence or awareness to determine if the participant should continue or discontinue the fusion polypeptide described herein.

Adherence to the study design requirements, including those specified in the SoA, is essential and required for study conduct.

All screening evaluations must be completed and reviewed to confirm that potential participants meet all eligibility criteria. The Investigator maintains a screening log to record details of all participants screened and to confirm eligibility or record reasons for screening failure, as applicable.

Procedures conducted as part of the participant's routine clinical management (e.g., blood count) and obtained before signing of the ICF may be utilized for screening or baseline purposes provided the procedures met the protocol-specified criteria and were performed within the time frame defined in the SoA, if consistent with site standard operating procedures.

### 8.1 Efficacy Assessments

Timing for collection of assessments for Cohort 1 is detailed in Section 1.3, Table 3, and Table 6. Timing for intensive collection of PK and PD samples for Cohorts 1 and 2 is detailed in Section 1.3, Table 5. Timing for collection of assessments for Cohort 2 are detailed in Section 1.3, Table 4 and Table 6. If conducted, timing for collection of assessments for optional Cohort 3 are detailed in Table 7 and Table 8.

### 8.1.1 Change in Complement Biomarkers

The following complement markers (absolute and percentage change from baseline) is measured during the study:
complement component Ba (Ba)
complement component C3a (C3a)
soluble complement component C5B-9 (sC5B9)
   Other complement markers may be evaluated, if feasible.

### 8.1.2 Change in Biomarkers Related to VOC

The following markers of VOC (absolute and percentage change from baseline) may be measured during the study:
Hemopexin
Nitric oxide
Inflammatory markers (e.g., interleukin-1)
Cell adhesion markers (e.g., soluble P-selectin)
   Other markers may be evaluated, if feasible.

### 8.1.3 Change in Hemoglobin

Blood samples are collected for assessment of hemoglobin changes from baseline.

### 8.1.4 Markers of Hemolysis

The following markers of hemolysis are measured during the study:
Serum LDH levels
Absolute reticulocyte count
Serum indirect bilirubin
Serum haptoglobin and hemopexin

### 8.1.5 Assessment of VOC

Sickle cell disease-related pain crises (VOC) are collected throughout the study per detailed in the SoA (Section 1.3).

Patients are issued a paper diary to record symptoms of VOC after signing the informed consent. The diary is collected by the Investigator or designee at every visit to the site and a new diary is provided.

### 8.2 Safety Assessments

Planned time points for all safety assessments for all cohorts are provided in the SoA (Section 1.3).

### 8.2.1 Physical Examination

A complete physical examination includes, at a minimum, assessments of the general appearance; skin; head, ears, eyes, nose, and throat; neck; lymph nodes; chest; heart; abdominal cavity; limbs; central nervous system; and musculoskeletal system.

An abbreviated physical examination includes, at a minimum, assessments of the skin, lungs, cardiovascular system, and abdomen (liver and spleen).

Height, weight and BMI are recorded per Section 1.3, SoA.

Investigators should pay special attention to clinical signs related to previous serious illnesses.

### 8.2.2 Vital Signs

Vital sign measurements are taken after the participant has been resting in the supine or semi-recumbent position for at least 5 minutes and include temperature (tympanic or oral), respiratory rate, supine blood pressure, and pulse. Ideally, the same arm for each participant should be used for BP and pulse measurements. Orthostatic (standing) blood pressure is only measured at Screening.

The timing of vital sign measurements is described in the SoA (Section 1.3).

Out of range blood pressure or pulse measurements is repeated at the Investigator's discretion. Confirmed, clinically significant vital sign measurements is recorded as AEs.

### 8.2.3 Electrocardiograms

Triplicate 12-lead ECGs are recorded at the time points described in the SoA (Section 1.3) to obtain heart rate, PR, QRS, and QT intervals. 12-lead ECG recordings are made after the participants have been resting in a supine position for at least 10 minutes.

At each time point at which triplicate ECGs are required, 3 individual ECG tracings should be obtained as closely as possible in succession but no more than 2 minutes apart.

8.2.3.1 Safety Review of 12-lead Electrocardiograms

All recorded ECGs are reviewed by the Investigator or qualified designee. If a participant shows an abnormal ECG, additional safety recordings may be made, and the abnormality is followed to resolution.

### 8.2.4 Clinical Laboratory Assessments

See Table 13 for the list of clinical laboratory tests to be performed and to the SoA (Section 1.3) for the timing and frequency. Clinical laboratory assessments are performed by a central laboratory unless otherwise specified.

The Investigator must review the laboratory report, document this review, and record any clinically relevant changes occurring during the study in the AE section of the eCRF. The laboratory reports must be filed with the source documents. Clinically significant abnormal laboratory findings are those which are not associated with the underlying disease, unless judged by the Investigator to be more severe than expected for the participant's condition.

All laboratory tests with values considered clinically significantly abnormal during participation in the study should be repeated until the values return to normal or baseline or are no longer considered clinically significant by the Investigator or Medical Monitor.
If such values do not return to normal/baseline within a period of time judged reasonable by the Investigator, the etiology should be identified, and the Investigator notified.
All protocol-required laboratory assessments must be conducted in accordance with the Laboratory Manual and the SoA.

If laboratory values from non-protocol specified laboratory assessments performed at the institution's local laboratory require a change in participant management or are considered clinically significant by the Investigator (e.g., SAE or AE or dose modification), then the results must be recorded in the eCRF.

### 8.2.5 Clinical Safety Laboratory Assessments

### 8.2.5.1 Virus Serology

Blood samples collected at Screening are analyzed for HIV-1, HIV-2, HbsAg, HBc antibody (anti-HBc IgG+IgM, if IgG positive), and HCV antibody titers.

### 8.2.6 Injection Site Evaluation

Subcutaneous injection site evaluations are performed at the time points specified in the SoA (Section 1.3). Injection site reactions are recorded as AEs unless deemed clinically significant.

### 8.2.7 Injection-associated Reactions

Injection-associated reactions are defined as systemic AEs (e.g., fever, chills, flushing, alterations in heart rate and blood pressure, dyspnea, nausea, vomiting, diarrhea, and generalized skin rashes) occurring during or within 24 hours of the start of SC injection that are assessed by the Investigator to be related to the study drug.

### 8.2.8 Vaccine and Antibiotic Prophylaxis

To mitigate the risk of N. meningitidis infection associated with complement inhibition, participants are administered the following:
1. Patients are vaccinated with MCV4 and serogroup B meningococcal vaccinations, if available at least 14 days before dosing, if not already vaccinated within 3 years before the first dose (or per national/local guidelines).
2. Patients are treated with prophylactic antibiotics at the Investigator's discretion.
   Patients must be vaccinated against other pathogens (e.g., Haemophiles influenzae, Streptococcus pneumoniae) according to current national/local guidelines.

### 8.3 Adverse Events and Serious Adverse Events

All AEs are reported to the Investigator or qualified designee by the participant (or, when appropriate, by a caregiver, surrogate, or the participant's legally authorized representative).

The Investigator and any qualified designees are responsible for detecting, documenting, and recording events that meet the definition of an AE or SAE and remain responsible for following up AEs that are serious, considered related to the study intervention or study procedures, or that caused the participant to discontinue the study intervention (see Section 7).

### 8.3.1 Time Period and Frequency for Collecting AE and SAE Information

All AEs and SAEs are collected from the signing of the ICF until the last follow-up visit. All SAEs are recorded and reported to the Investigator or the designee immediately and under no circumstance should this exceed 24 hours. The Investigator submits any updated SAE data to the Investigator within 24 hours of the date the investigational site became aware of the event.

Investigators are not obligated to actively seek AE or SAE data after conclusion of the study participation. However, if the Investigator learns of any SAE, including a death, at any time after a participant has been discharged from the study, and he/she considers the event to be reasonably related to the study intervention or study participation, the Investigator must promptly notify the Investigator.

### 8.3.2 Method of Detecting AEs and SAEs

Care is taken not to introduce bias when detecting AEs and/or SAEs. Open-ended and non-leading verbal questioning of the participant is the preferred method to inquire about AE occurrences.

### 8.3.3 Follow-Up of AEs and SAEs

After the initial AE/SAE report, the Investigator is required to proactively follow up on each participant at subsequent visits/contacts. All SAEs are followed up until resolution, stabilization, the event is otherwise explained, or the participant is lost to follow-up (as defined in Section 7.4).

### 8.3.4 Regulatory Reporting Requirements for SAEs

Prompt notification of an SAE by the Investigator to the Investigator is essential so that legal obligations and ethical responsibilities towards the safety of participants and the safety of a study intervention under clinical investigation are met.

The Investigator has a legal responsibility to notify both the local regulatory authority and other regulatory agencies about the safety of a study intervention under clinical investigation. The Investigator complies with country-specific regulatory requirements relating to safety reporting to the regulatory authority, Institutional Review Boards (IRBs)/IECs, and Investigators.

Suspected unexpected serious adverse reactions must be reported according to local regulatory requirements and forwarded to Investigators as necessary.

An Investigator who receives an Investigator safety report describing an SAE or other specific safety information (e.g., summary or listing of SAEs) from the Investigator reviews and then files it along with the IB and notifies the IRB/IEC, if appropriate according to local requirements.

### 8.4 Treatment of Overdose

For this study, any dose of study intervention greater than that specified in the protocol is considered an overdose. There is no specific treatment or antidote for overdose.

Overdoses are medication errors that are not considered AEs unless there is an untoward medical occurrence resulting from the overdose.

In the event of an overdose or suspected overdose, the Investigator should:
1. Contact the Medical Monitor immediately.
2. Closely monitor the participant for any AE/SAE.
3. Obtain a sample for PK/PD analysis if requested by the Medical Monitor (determined on a case-by-case basis).
4. Document the quantity of the excess dose as well as the duration of the overdose in the eCRF.

Decisions regarding dose interruptions or modifications are made by the Investigator in consultation with the Medical Monitor based on the clinical evaluation of the participant.

### 8.5 Pharmacokinetics

Whole blood samples are collected for measurement of serum concentrations of the fusion polypeptide described herein as specified in the SoA (Section 1.3). Additional samples may be collected at additional time points during the study if warranted and agreed upon with the Investigator. The total blood volume does not exceed the volume limit for participants per national/local guidelines. The timing of sampling may be altered during the course of the study, based on newly available data (e.g., to obtain data closer to the time of peak serum concentrations) to ensure appropriate monitoring.
Instructions for the collection and handling of biological samples are provided by the Investigator. The actual date and time (24-hour clock time) of each sample is recorded.
Samples are used to evaluate the PK of the fusion polypeptide described herein. Samples collected for analyses of the fusion polypeptide described herein serum concentration may also be used to evaluate safety or efficacy aspects related to concerns arising during or after the study.
Samples may be used for research to develop methods, assays, prognostics and/or companion diagnostics related to dysregulated complement activity.

### 8.6 Pharmacodynamics

Whole blood samples are collected for measurement of serum total and free properdin concentrations, CAP activity, and potentially other measures of complement activation as specified in the SoA (Section 1.3). Additional samples may be collected at additional time points during the study if warranted and agreed upon with the Investigator, and upon receipt of consent from the study participant. The total blood volume does not exceed the blood volume limit for participants per national/local guidelines. The timing of sampling may be altered during the course of the study based on newly available data (e.g., to obtain data closer to the time of peak plasma concentrations) to ensure appropriate monitoring.

Instructions for the collection and handling of biological samples are provided by the Investigator. The actual date and time (24-hour clock time) of each sample is recorded.

Samples are used to evaluate the PD of the fusion polypeptide described herein. Samples collected for analyses of the fusion polypeptide described herein concentration may also be used to evaluate safety or efficacy aspects related to concerns arising during or after the study. Unused samples may be retained for a period of up to 25 years to perform additional assessments as necessary.

### 8.7 Genetics

Genetics are not evaluated in this study.

### 8.8 Biomarkers

Collection of samples for biomarker research (e.g., exploratory) is also part of this study.

The following samples for biomarker research are required and are collected from all participants in this study as specified in the SoA (Section 1.3):
Blood
Urine
   Samples are collected for testing that may include, but are not limited to, markers of complement dysregulation, inflammation, and endothelial activation/damage.

### 8.9 Immunogenicity Assessments

Antibodies to the fusion polypeptides (ADAs) are evaluated in whole blood samples collected from all participants according to the SoA (Section 1.3).

Serum samples are screened for ADAs. If the screen is positive, the sample is analyzed using a confirmatory ADA assay and the titer of confirmed positive samples is reported. The detection and characterization of antibodies to the fusion polypeptide described herein is performed using a validated assay method by or under the supervision of the Investigator. Samples may be further characterized to determine the titer and the presence of neutralizing antibodies (as an exploratory analysis) if deemed necessary.

The actual date and time (24-hour clock time) of each sample is recorded. Samples may be banked for a period of up to 25 years in order to perform additional safety assessments, as necessary.

Detailed instructions on the procedure for collecting, processing, storing, and shipping serum samples for immunogenicity analysis are provided in the laboratory manual.

### 9. Statistical Considerations

### 9.1 Statistical Hypotheses

Not applicable.

### 9.2 Sample Size Determination

Twelve participants are enrolled in each of Cohorts 1 and 2. In patients with stable SCD, hemoglobin level is unlikely to change. The sample size is determined based on a targeted hemoglobin change from baseline to exclude 0 g/dL with a lower 2-sided 95% confidence bound. Assuming a standard deviation of 1 g/dL, a sample size of 12 participants provides 88% power to detect a change from baseline of 1 g/dL with a 2-sided significance level of 0.05. In the optional Cohort 3, 6 participants are enrolled to define the exposure/response relationship of the fusion polypeptide described herein by combining Cohort 1, Cohort 2 and Cohort 3 data using a PK/PD modeling approach. The Cohort 3 sample size is not determined for power purposes.

### 9.3 Populations for Analyses

For purposes of analysis, the following populations are defined:

| Population | Description |
|---|---|
| Safety | All participants who receive at least 1 dose of study drug. |
| Enrolled Set | All subjects who sign the ICF and passed inclusion/ |
| | exclusion criteria. |
| Full Analysis | All subjects in the Enrolled Set who have been randomized |
| Set (FAS) | and received at least 1 dose of study drug. |
| Per Protocol | All subjects in the Enrolled Set without any major protocol |
| Set (PPS) | deviation. |
| Pharma- | All participants who receive at least 1 dose of the study |
| cokinetic | drug and have at least 1 post-dose PK sample. |
| Pharma- | All participants who receive at least 1 dose of study drug |
| codynamic | and who have evaluable properdin concentration, CAP or |
| | CCP activity data. |
| Immuno- | All participants who have a predose and at least 1 postdose |
| genicity | ADA sample collected. |

Abbreviations: ADA = antidrug antibody; CAP = complement alternative pathway; CCP = complement classical pathway; ICF = informed consent form; PK = pharmacokinetic(s).

### 9.4 Statistical Analyses

In general, descriptive statistics for continuous variables includes number of non-missing values, arithmetic mean, standard deviation, median, minimum, and maximum. Descriptive statistics for PK parameters include number of observations, arithmetic mean, standard deviation, arithmetic coefficient of variation (% CV), median, minimum, maximum, geometric mean, and geometric % CV. Categorical variables are summarized using percentages and frequency counts, by cohort and time point.

A SAP is developed and finalized before first data cutoff/database lock and further describes the participant populations to be included in the analyses, and procedures for accounting for missing, unused, and spurious data as appropriate. This section is a high-level summary of the planned statistical analyses of the primary and secondary endpoints.

### 9.4.1 Efficacy Analyses

### 9.4.1.1 Change in Complement Biomarkers

Absolute and percentage of change from baseline in complement biomarkers (e.g., Ba, C3a and sC5B9) are evaluated at the end of treatment (12 weeks) for Cohorts 1 and 2. Additional details are described in the SAP.

### 9.4.1.2 Change in Hemoglobin

Absolute and percentage change from baseline in hemoglobin are evaluated at the end of treatment (12 weeks) for Cohorts 1 and 2. Additional details are described in the SAP.

### 9.4.1.3 Time to Hemoglobin Response

Hemoglobin response is defined as an increase in hemoglobin levels of >1 g/dL from baseline. Hemoglobin response is evaluated at the end of treatment (12 weeks) for Cohorts 1 and 2. Additional details are described in the SAP.

### 9.4.1.4 Markers of Hemolysis

Absolute and percentage change from baseline of the markers of hemolysis (serum LDH levels, absolute reticulocyte count, serum indirect bilirubin, serum haptoglobin and hemopexin) are evaluated at the end of treatment (12 weeks) for Cohorts 1 and 2. Additional details are described in the SAP.

### 9.4.1.5 Exploratory Analysis of Change in Biomarkers Related to VOC

Biomarkers related to VOC may be evaluated after end of treatment (12 weeks for Cohorts 1 and 2). Additional details are described in the SAP.

### 9.4.2 Exploratory Assessment of VOC

Sickle cell disease-related pain crises (VOC) are defined as acute episodes of pain, with no medically determined cause other than a VOC event that result in a medical facility visit and treatment with either oral or parenteral narcotic agents, or with a parenteral nonsteroidal anti-inflammatory drug. Uncomplicated VOCs are defined as no occurrence of any other SCD complication during the VOC episode. Complicated VOCs are defined as the presence of a diagnosis of other SCD complications during the VOC episode. Acute chest syndrome, hepatic sequestration, splenic sequestration, and priapism are considered as VOC events in this study. Complicated VOCs are also to be reported as AEs. The following may also be assessed if available:
Number of VOCs leading to a healthcare visit
Number of uncomplicated VOCs, acute chest syndrome, hepatic sequestration, splenic sequestration, and priapism
Time to first VOC after first dose of study drug

### 9.4.3 Safety Analyses

The primary endpoint for the study is safety and tolerability.

All safety analyses are performed on the Safety Population and are reported by each cohort.

Safety analyses include an analysis of all TEAEs, ECGs, clinical laboratory data, physical examinations, and vital sign measurements using descriptive statistics. No inferential statistical analyses are planned on the safety parameters of this study. The prevalence of AEs and SAEs is summarized, by SOC and Preferred Term for each cohort and treatment arm and overall, within each treatment arm, and by relationship to study drug. AEs also are summarized by cohort and treatment arm, and overall, within each treatment arm, and by severity. SAEs and AEs resulting in withdrawal from the study are listed. Participants having multiple AEs within a category (e.g., overall, SOC, Preferred Term) are counted once in that category. For severity tables, a participant's most severe event within a category is counted.

Changes from baseline in vital sign measurements and laboratory assessments (e.g., clinical chemistry, cell blood count with differential, and urinalysis) are summarized by each cohort and overall. Laboratory parameter values are graded according to the Common Terminology Criteria for Adverse Events (CTCAE, v5.0, published 27 Nov. 2017). Shift tables by cohort and treatment arm are produced for these laboratory parameters. These tables summarize the number of participants with each baseline grade relative to the reference ranges and changes to the worst highest grade assessed postdose during the study.

All concomitant medications are coded using the World Health Organization Drug Dictionary, and the frequency and percentage of concomitant medications is summarized.

### 9.4.3.1 ECG Analysis

Cardiac assessments are performed in the Safety Population.

The ECG parameters are measured at the specified time points, including heart rate, PR, RR, QRS, QT, and QTcF intervals. The average of the triplicate ECG readings at the time points collected is calculated, and changes from pretreatment baseline values are assessed by each cohort and treatment arm.

An outlier analysis is performed that summarize the absolute count, frequency and percentage of participants who meet any of the following outlier criteria at each visit by cohort and treatment arm:
QT, QTcF interval >450 msec
QT, QTcF interval >480 msec
QT, QTcF interval >500 msec
QT, QTcF interval increases from baseline >30 msec
QT, QTcF interval increases from baseline >60 msec
   Analysis of drug-related QT/QTc interval changes relative to plasma PK concentrations may be conducted on all dose regimens. The principles of this analysis follow the statistical methods described by Garnett et al. (Garnett, 2018).

Detailed analyses are specified in the SAP or a separate ECG analysis plan.

### 9.4.4 Other Analyses

### 9.4.1.1 Pharmacokinetic Analyses

All PK analyses are performed on the PK Population and are reported by cohort.

The individual serum concentration data from patients who receive the SC dose of the fusion polypeptide described herein with actual sampling dates and times are used to characterize PK using a population PK analysis approach. The details are provided in the SAP.

### 9.4.1.2 Pharmacodynamic Analyses

All PD analyses are performed on the PD Population and are reported by cohort.

The PD effects of all the SC doses of the fusion polypeptide described herein administered are evaluated by assessing changes in serum total and free properdin concentrations and CAP activity using the Weislab AP assay. In addition, an exploratory assessment of other measures of properdin activity overtime may be considered as deemed appropriate.

### 9.4.1.3 Immunogenicity Analysis

For assessment of immunogenicity, the incidence of confirmed positive ADAs is summarized. Additionally, following confirmation of positive ADAs, samples are assessed for ADA titer and presence of neutralizing antibodies (if possible).

### 9.4.1.4 Exploratory Analysis

Additional exploratory analysis on biomarker assays and clinical efficacy endpoint may be conducted. Details of these analyses are presented in the SAP.

### 9.5 Interim Analyses

An interim analysis may be performed after at least 12 patients from Cohorts 1 and 2 (6 from each cohort) have enrolled and completed the Treatment Period to inform later phase trials. The interim analysis includes safety, efficacy, PK/PD and immunogenicity data. Details of this analysis are presented in the SAP.

### 10. Laboratory Tests

The protocol-required clinical laboratory tests detailed in Table 13 are performed by a central laboratory unless otherwise specified.

TABLE 13
Protocol-required Laboratory Assessments

| Laboratory | |
|---|---|
| Assessments | Parameters |
| Hematology | Platelet count |
| | Red blood cell (RBC) count |
| | Hemoglobin |
| | Free Hemoglobin |
| | Hemoglobinopathy test (e.g., hemoglobin electrophoresis, |
| | screening only) |
| | Hematocrit |
| | Haptoglobin |
| | Hemopexin |
| | RBC indices (MCV, MCH, % reticulocytes) |
| | White blood cell (WBC) count with differential |
| | (neutrophils, lymphocytes, monocytes, eosinophils, |
| | basophils) |
| Clinical | Alanine aminotransferase (ALT)/serum glutamic-pyruvic |
| chemistry | transaminase (SGPT) |
| | Alkaline phosphatase |
| | Aspartate aminotransferase (AST)/serum glutamic- |
| | oxaloacetic transaminase (SGOT) |
| | Blood urea nitrogen (optional) |
| | Calcium |
| | Creatinine |
| | Creatine kinase |
| | Hemoglobin A1C |
| | Fructosamine |
| | LDH |
| | Potassium |
| | Sodium |
| | Serum albumin |
| | Total and direct bilirubin |
| | Indirect bilirubin |
| | Total protein |
| | Urea |
| Coagulation | Prothrombin time, partial thromboplastin time, international |
| Routine | normalized ratio Specific gravity |
| urinalysis | pH, glucose, protein, blood, ketones, bilirubin, |
| | urobilinogen, nitrite, leukocyte esterase by dipstick |
| | Microscopic examination (If blood or protein is abnormal |
| Spot urine | Protein, albumin, creatinine, and protein-to-creatinine and |
| studies | albumin/creatinine ratio |
| Complement | Change in CAP activity using the Wieslab AP assay |
| activity | |
| Biomarkers | Complement biomarkers |
| | Biomarkers related to VOC |
| | Other biomarkers may be considered if feasible |
| Other | Serum human chorionic gonadotropin (hCG) pregnancy test |
| screening | (as needed for women of childbearing potential) ^{a} |
| tests | Follicle-stimulating hormone and estradiol (as needed in |
| | women of non-childbearing potential only) |
| | Human immunodeficiency virus (HIV)-1 and HIV-2 |
| | antibodies, hepatitis B surface antigen (HbsAg), anti-HBc |
| | IgG + IgM (if IgG positive) and hepatitis C virus |
| | antibodies (anti-HCV) |

^{a} Local urine testing is standard for the protocol unless serum testing is required by local regulation or IRB/IEC.
Abbreviations: AP = alternative pathway; CAP = complement alternative pathway; CCP = complement classical pathway; HBc = hepatitis B core; IEC = Independent Ethics Committee; IgG = immunoglobulin G; IgM = immunoglobulin M; IRB = Institutional Review Board; LDH = lactate dehydrogenase; LP = lectin pathway; MCH = mean corpuscular hemoglobin; MCV = mean corpuscular volume; SAE = serious adverse event; VOC = vaso-occlusive crisis.

TABLE 14
Abbreviations and Specialist Terms

| Abbreviation | |
|---|---|
| or Term | Explanation |
| ADA | antidrug antibody |
| AE | adverse event |
| AP | alternative pathway |
| AUC₀₋₁₆₈ | area under the concentration-time curve from time |
| | 0 to 168 hours |
| Ba | complement component Ba |
| BMI | body mass index |
| C3 | complement component C3 |
| C3a | complement component C3a |
| C5 | complement component 5 |
| C9 | complement component 9 |
| CAP | complement alternative pathway |
| CFR | Code of Federal Regulations |
| CIOMS | Council for International Organizations of Medical Sciences |
| Cₘₐₓ | maximum observed serum concentration |
| COVID-19 | coronavirus disease 2019 |
| CTCAE | Common Terminology Criteria for Adverse Events |
| % CV | coefficient of variation |
| DMC | Data Monitoring Committee |
| EC | ethics committee |
| ECG | electrocardiogram |
| eCRF | electronic case report form |
| EDC | electronic data capture |
| EOS | end of study |
| FIH | first-in-human |
| FSH | follicle-stimulating hormone |
| GCP | Good Clinical Practice |
| GDS | Global Drug Safety |
| GDPR | General Data Protection Regulation |
| GLP | Good Laboratory Practice |
| HBC | hepatitis B core |
| HbS | sickle hemoglobin |
| HbsAg | hepatitis B surface antigen |
| HCV | hepatitis C virus |
| HiB | Haemophilus influenzae type b |
| HIPAA | Health Insurance Portability and Accountability Act |
| HIV | human immunodeficiency virus |
| HRT | hormonal replacement therapy |
| IB | Investigator's Brochure |
| ICF | informed consent form |
| ICH | International Council for Harmonisation of Technical |
| | Requirements for Pharmaceuticals for Human Use |
| IEC | Independent Ethics Committee |
| IRB | Institutional Review Board |
| IV | intravenous(ly) |
| K_{D} | dissociation constant |
| LDH | lactate dehydrogenase |
| mAb | monoclonal antibody |
| MCV4 | tetravalent meningococcal conjugate vaccine |
| NOAEL | no observed adverse effect level |
| PD | pharmacodynamic(s) |
| PK | pharmacokinetic(s) |
| QTcF | QT interval corrected using the Fridericia's formula |
| Q2W | once every 2 weeks |
| Q4W | once every four weeks |
| QW | once weekly |
| RBC | red blood cell |
| SAE | serious adverse event |
| SAP | statistical analysis plan |
| SC | subcutaneous(ly) |
| sC5b9 | soluble complement component C5b-9 |
| SCD | sickle cell disease |
| SoA | schedule of activities |
| SOC | System Organ Class |
| TCR | tissue cross reactivity |
| TEAE | treatment-emergent adverse event |
| VHH | single variable domain on a heavy chain antibody |
| VOC | vaso-occlusive crisis |
| WOCBP | woman of childbearing potential |

### Example 2. Safety, Tolerability, Pharmacokinetics, Pharmacodynamics, and Immunogenicity of Anti-Properdin/Anti-Serum Albumin Bispecific Single Variable Domain Antibody in Healthy Adults: Results of a Phase 1 Study

This randomized, double-blind, placebo-controlled evaluated the safety, tolerability, pharmacokinetics (PK), pharmacodynamics (PD), and immunogenicity of subcutaneous (SC) and intravenous (IV) administration the fusion polypeptide having the amino acid sequence of SEQ ID NO:1, or a modification thereof, in healthy adults.

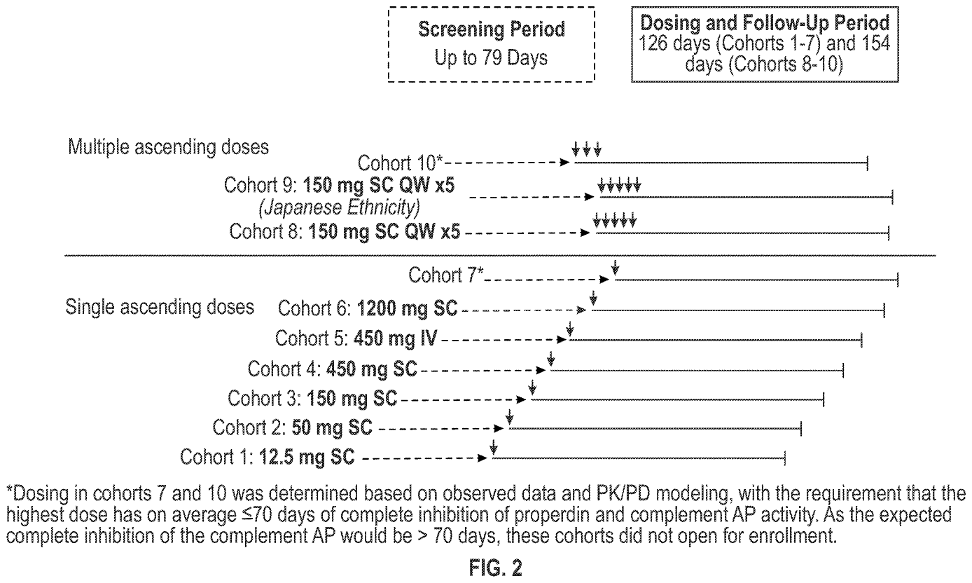

In this phase I first-in-human (FIH) study, healthy volunteers aged 18 to 65 years were randomized to the fusion polypeptide or placebo (3:1). The fusion polypeptide was administered SC in single and multiple ascending doses and IV as a single dose. Ten dosing cohorts were planned (FIG. 2). Two cohorts did not open for enrollment, as the expected complete inhibition of the complement AP would be >70 days. Due to the potentially increased risk of Neisseria meningitidis infection with properdin inhibition, vaccination was required before receiving the fusion polypeptide described herein and the participants remained on antibiotics for the duration of treatment.

Sixty participants were randomized (fusion polypeptide, 45; placebo, 15). Cohorts 1, 3, 4, and 5 had 5 participants (each) treated with the fusion polypeptide; cohorts 2, 6, and 9 had 6 participants treated with the fusion polypeptide; and cohort 8 had 7 participants treated with the fusion polypeptide. Demographic and baseline characteristics were similar for the fusion polypeptide and placebo arms. Four participants discontinued from the study: 1 was lost to follow-up, 1 after use of prohibited medication, and 2 withdrew consent (1 following an adverse event [AE] on placebo). There were no discontinuations due to AEs in participants exposed to the fusion polypeptide. The majority of treatment-emergent AEs (TEAEs) were mild and assessed as not related to study treatment. There were no serious AEs, serious events of infections, or deaths. TEAE incidence was similar across cohorts. Treatment-related TEAEs occurred more frequently in the fusion polypeptide than the placebo group (11 [24.4%] vs 2 [13.3%]); the most common treatment-related TEAEs with the fusion polypeptide were nausea (n=2), headache (n=2), and infusion site erythema (n=2). No infections with N. meningitidis were reported.

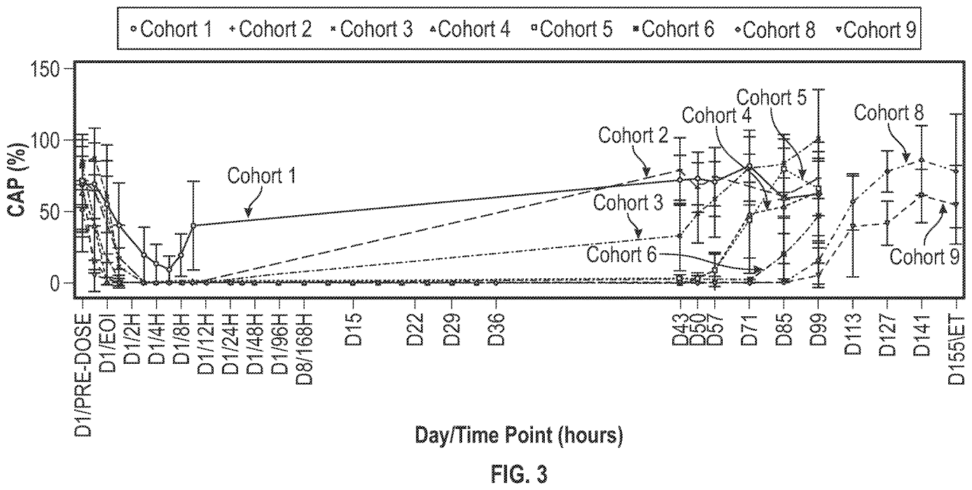

Dose proportionality was observed for all single dose cohorts except 50 mg. Geometric mean accumulation ratios, R_{Cmax} and R_{AUC}, were approximately 2.8 and 3, respectively, after multiple doses. Mean absolute bioavailability of the SC administered fusion polypeptide was 94%. Complement alternative pathway (AP) activity decreased immediately following administration of the fusion polypeptide (FIG. 3). With increasing dose, the duration of complete AP blockade (<1% of baseline) increased; in cohorts 4, 5, and 6, complete AP inhibition was sustained for >30 days. In all single-dose cohorts, mean % AP hemolysis returned to baseline by the end of the observation period. In multiple-dose cohorts, complete inhibition of AP activity was observed for 70 days and 84 days (cohorts 8 and 9, respectively). There was no change in the classical pathway (CP) and lectin pathway (LP) activity.

Four participants had pre-existing immunoreactivity at baseline, none of which were treatment-boosted. Treatment-emergent antidrug antibodies (ADA) occurred in 20%, 40%, 100%, and 83.3% of participants in the 150 mg SC, 450 mg SC, 450 mg IV, and 1200 mg SC groups, respectively; most had low titers, which did not impact PK. In multiple-dose cohorts (cohorts 8 and 9), 71.4% and 100% developed ADA, respectively, most of whom had low titers and transient duration of response.

In this FIH study in healthy participants, the fusion polypeptide described herein showed no unexpected safety concerns and was well-tolerated. Complement AP inhibition by the fusion polypeptide described herein was rapid and complete, and the CP and LP were not affected.

### Other Embodiments

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method for treating a human patient with sickle cell disease, the method comprising administering a properdin binding antibody or antigen-binding fragment thereof to the patient, wherein the properdin binding antibody or an antigen binding fragment thereof comprises CDR1, CDR2, and CDR3 sequences as set forth in SEQ ID NOs: 2, 3, and 4, respectively.

2. The method of claim 1, wherein the antibody or antigen binding fragment thereof further comprises a human serum albumin binding sequence.

3. The method of claim 2, wherein the human serum albumin binding sequence is fused to the C-terminus of the properdin binding antibody or antigen-binding fragment thereof.

4. The method of claim 3, wherein the human serum albumin binding sequence is fused to the C-terminus of the properdin binding antibody or antigen-binding fragment thereof by linker.

5. The method of claim 4, wherein the linker comprises the amino acid sequence of SEQ ID NO: 10.

6. The method of any one of claims 2-5, wherein the human serum albumin binding sequence comprises CDR1, CDR2, and CDR3 sequences as set forth in SEQ ID NOs: 5, 6, and 7.

7. The method of any one of claims 1-6, wherein the antibody or antigen binding fragment thereof comprises the sequence of SEQ ID NO: 1, or a modification thereof.

8. The method of claim 7, wherein the modification comprises conversion of the N-terminal glutamine of the sequence of SEQ ID NO: 1 to pyro-glutamate.

9. The method of any one of claims 1-8, wherein the antibody or the antigen binding fragment thereof is administered to the patient at a dose of 300 mg.

10. The method claim 9, wherein the antibody or the antigen binding fragment thereof administered to the patient weekly.

11. The method of claim 9 or 10, where the antibody or the antigen binding fragment thereof is administered to the patient for up to 12 weeks.

12. The method of claim 9, wherein the antibody or the antigen binding fragment thereof once is administered to the patient every 2 weeks.

13. The method of claim 12, wherein the antibody or the antigen binding fragment thereof is administered up to 4 times.

14. The method of any one of claims 1-8, wherein the antibody or the antigen binding fragment thereof is administered to the patient at a dose of 600 mg.

15. The method of claim 14, wherein the antibody or the antigen binding fragment thereof is administered to the patient every 4 weeks.

16. The method of claim 14 or 15, wherein the antibody or the antigen binding fragment thereof is administered the patient up to 4 times.

17. A method for treating a human patient with sickle cell disease, the method comprising administering a properdin binding antibody or antigen-binding fragment thereof to the patient, wherein the properdin binding antibody or an antigen binding fragment thereof comprises the sequence of SEQ ID NO: 1, or a modification thereof, and wherein the antibody or the antigen binding fragment thereof is administered to the patient at a dose of 300 mg weekly for up to 12 weeks.

18. A method for treating a human patient with sickle cell disease, the method comprising administering a properdin binding antibody or antigen-binding fragment thereof to the patient, wherein the properdin binding antibody or an antigen binding fragment thereof comprises the sequence of SEQ ID NO: 1, or a modification thereof, and wherein the antibody or the antigen binding fragment thereof is administered to the patient at a dose of 300 mg every 2 weeks up to 4 times.

19. A method for treating a human patient with sickle cell disease, the method comprising administering a properdin binding antibody or antigen-binding fragment thereof to the patient, wherein the properdin binding antibody or an antigen binding fragment thereof comprises the sequence of SEQ ID NO: 1, or a modification thereof, and wherein the antibody or the antigen binding fragment thereof is administered to the patient at a dose of 600 mg every 4 weeks up to 4 times.

20. The method of any one of claims 17-19, wherein the modification comprises conversion of the N-terminal glutamine of the sequence of SEQ ID NO: 1 to pyro-glutamate.

21. The method of any one of claims 1-20, wherein the patient has been clinically diagnosed with sickle cell disease.

22. The method of claim 21, wherein the sickle cell disease is HbSS or HbSβ⁰-thalassemia.

23. The method of any one of claims 1-22, wherein the patient is further administered hydroxyurea.

24. The method of claim 23, wherein the patient has been receiving a stable dose of hydroxyurea for at least 3 months prior to administration the antibody or the antigen binding fragment thereof.

25. The method of any one of claims 1-22, wherein the patient has not been administered hydroxyurea for at least 30 days prior to administration the antibody or the antigen binding fragment thereof.

26. The method of any one of claims 1-25, wherein the patient experiences no treatment emergent adverse events after 12 weeks of treatment.

27. The method of any one of claims 1-26, wherein the patient experiences no serious adverse events after 12 weeks of treatment.

28. The method of any one of claims 1-26, wherein the patient experiences no adverse events after 12 weeks of treatment.

29. The method of any one of claims 1-28, further comprising measuring a change in serum concentration of the antibody or antigen binding fragment thereof for up to 30 weeks after starting treatment.

30. The method of any one of claims 1-29, further comprising measuring a change in blood concentration of an anti-drug antibody for up to 30 weeks after starting treatment.

31. The method of any one of claims 1-30, wherein the patient experiences a change in serum concentration from baseline of total and free properdin after up to 30 weeks of treatment.

32. The method of any one of claims 1-31, wherein the patient experiences a change in serum concentration from baseline of complement component Ba (Ba), complement component C3a (C3a), or soluble complement component C5B-9 (sC5B9) after 12 weeks of treatment.

33. The method of any one of claims 1-32, wherein the patient experiences a change from baseline in blood or serum concentration of hemopexin, nitric oxide, an inflammatory marker, or a cell adhesion marker after 12 weeks of treatment.

34. The method of claim 33, wherein the inflammatory marker comprises interleukin-1.

35. The method of claim 33 or 34, wherein the cell adhesion marker comprises soluble P-selectin.

36. The method of any one of claims 1-30, wherein the patient experiences a change from baseline in hemoglobin levels after 12 weeks of treatment.

37. The method of any one of claims 1-36, wherein the patient experiences a change in serum LDH levels, indirect bilirubin, haptoglobin, or hemopexin after 12 weeks from baseline.

38. The method of any one of claims 1-37, wherein the patient experiences a change in reticulocyte levels after 12 weeks from baseline.

39. The method of any one of claims 1-38, wherein the patient experiences a reduced rate of vaso occlusive crisis after 12 weeks of treatment in comparison to baseline.

40. The method of any one of claims 1-39, wherein the patient experiences an increased time to first vaso-occlusive crisis after 12 weeks of treatment in comparison to baseline.

41. The method of any one of claims 1-40, wherein the antibody or antigen binding fragment thereof is formulated for subcutaneous administration.

42. The method of any one of claims 1-41, wherein the antibody or antigen binding fragment thereof is formulated at a pH of 5.4 at a concentration of 150 mg/mL in an aqueous solution comprising 20 nM sodium acetate, 250 mM sucrose, and 0.05% polysorbate-80.

43. The method of any one of claims 1-42, wherein the human patient is between 18 and 65 years of age.

44. The method of any one of claims 1-43, wherein the human patient has a body weight of 40 kg.


SEQUENCE LISTING (57 sequences)

SEQ ID NO: 1 - protein, 256 aa, synthetic construct
QVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGEGGGGEGGGGEVQLLESGGGLVQPGGSLRLSCAASGRISSIIHMAWFRQAPGKERELVSEISRVGTTVYADSVKGRFTISRDNSKNTLYLQMNSLKPEDTAVYYCNALQYEKHGGADYWGQGTLVTVSS

SEQ ID NO: 2 - protein, 10 aa, synthetic construct
GRISSIIHMA

SEQ ID NO: 3 - protein, 13 aa, synthetic construct
RVGTTVYADSVKG

SEQ ID NO: 4 - protein, 11 aa, synthetic construct
LQYEKHGGADY

SEQ ID NO: 5 - protein, 8 aa, synthetic construct
GRPVSNYA

SEQ ID NO: 6 - protein, 8 aa, synthetic construct
INWQKTAT

SEQ ID NO: 7 - protein, 17 aa, synthetic construct
AAVFRVVAPKTQYDYDY

SEQ ID NO: 8-9 - skipped (2 entries)

SEQ ID NO: 10 - protein, 15 aa, synthetic construct
GGGGEGGGGEGGGGE

SEQ ID NO: 11 - protein, 15 aa, synthetic construct
GGGGSGGGGSGGGGS

SEQ ID NO: 12 - protein, 5 aa, synthetic construct
GGGGS

SEQ ID NO: 13 - protein, 10 aa, synthetic construct
GGGGSGGGGS

SEQ ID NO: 14 - protein, 15 aa, synthetic construct
GGGGSGGGGSGGGGS

SEQ ID NO: 15 - protein, 20 aa, synthetic construct
GGGGSGGGGSGGGGSGGGGS

SEQ ID NO: 16 - protein, 25 aa, synthetic construct
GGGGSGGGGSGGGGSGGGGSGGGGS

SEQ ID NO: 17 - protein, 30 aa, synthetic construct
GGGGSGGGGSGGGGSGGGGSGGGGSGGGGS

SEQ ID NO: 18 - protein, 15 aa, synthetic construct
EAAAKEAAAKEAAAK

SEQ ID NO: 19 - protein, 5 aa, synthetic construct
PAPAP

SEQ ID NO: 20 - protein, 10 aa, synthetic construct
GGGGSPAPAP

SEQ ID NO: 21 - protein, 10 aa, synthetic construct
PAPAPGGGGS

SEQ ID NO: 22 - protein, 10 aa, synthetic construct
GGGDSGGGDS

SEQ ID NO: 23 - protein, 10 aa, synthetic construct
GGGESGGGES

SEQ ID NO: 24 - protein, 10 aa, synthetic construct
GGGDSGGGGS

SEQ ID NO: 25 - protein, 10 aa, synthetic construct
GGGASGGGGS

SEQ ID NO: 26 - protein, 10 aa, synthetic construct
GGGESGGGGS

SEQ ID NO: 27 - protein, 6 aa, synthetic construct
ASTKGP

SEQ ID NO: 28 - protein, 13 aa, synthetic construct
ASTKGPSVFPLAP

SEQ ID NO: 29 - protein, 4 aa, synthetic construct
GGGP

SEQ ID NO: 30 - protein, 8 aa, synthetic construct
GGGGGGGP

SEQ ID NO: 31 - protein, 9 aa, synthetic construct
PAPNLLGGP

SEQ ID NO: 32 - protein, 6 aa, synthetic construct
GGGGGG

SEQ ID NO: 33 - protein, 12 aa, synthetic construct
GGGGGGGGGGGG

SEQ ID NO: 34 - protein, 8 aa, synthetic construct
APELPGGP

SEQ ID NO: 35 - protein, 8 aa, synthetic construct
SEPQPQPG

SEQ ID NO: 36 - protein, 15 aa, synthetic construct
GGGSSGGGSSGGGSS

SEQ ID NO: 37 - protein, 14 aa, synthetic construct
GGGGGGGGGSGGGS

SEQ ID NO: 38 - protein, 15 aa, synthetic construct
GGGGSGGGGGGGGGS

SEQ ID NO: 39 - protein, 15 aa, synthetic construct
GGSSSGGSSSGGSSS

SEQ ID NO: 40 - protein, 15 aa, synthetic construct
GSSSSGSSSSGSSSS

SEQ ID NO: 41 - protein, 15 aa, synthetic construct
GGGGAGGGGSGGGGS

SEQ ID NO: 42 - protein, 15 aa, synthetic construct
GGGGSGGGGAGGGGS

SEQ ID NO: 43 - protein, 15 aa, synthetic construct
GGGASGGGGSGGGGS

SEQ ID NO: 44 - protein, 15 aa, synthetic construct
GGGGSGGGASGGGGS

SEQ ID NO: 45 - protein, 15 aa, synthetic construct
GGGGSAGGGSGGGGS

SEQ ID NO: 46 - protein, 15 aa, synthetic construct
GGGGSGGGGSAGGGS

SEQ ID NO: 47 - protein, 15 aa, synthetic construct
GGGGSAGGGSAGGGS

SEQ ID NO: 48 - protein, 15 aa, synthetic construct
GGGGDGGGGSGGGGS

SEQ ID NO: 49 - protein, 15 aa, synthetic construct
GGGGSGGGGDGGGGS

SEQ ID NO: 50 - protein, 15 aa, synthetic construct
GGGGDGGGGDGGGGS

SEQ ID NO: 51 - protein, 15 aa, synthetic construct
GGGGEGGGGSGGGGS

SEQ ID NO: 52 - protein, 15 aa, synthetic construct
GGGGSGGGGEGGGGS

SEQ ID NO: 53 - protein, 15 aa, synthetic construct
GGGGEGGGGEGGGGS

SEQ ID NO: 54 - protein, 15 aa, synthetic construct
GGGGAGGGGAGGGGS

SEQ ID NO: 55 - protein, 119 aa, synthetic construct
EVQLLESGGGLVQPGGSLRLSCAASGRISSIIHMAWFRQAPGKERELVSEISRVGTTVYADSVKGRFTISRDNSKNTLYLQMNSLKPEDTAVYYCNALQYEKHGGADYWGQGTLVTVSS

SEQ ID NO: 56 - protein, 123 aa, synthetic construct
QVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSS

SEQ ID NO: 57 - DNA, 774 bp, synthetic construct
  source 1..774: mol_type = other DNA
caggtgcagctggtggaaagcggcggaggcctggtcaagcctggcggcagcctgagactgagctgtgccgccagcggcagacccgtgtccaattacgccgctgcctggttccggcaggcccctggcaaagagagagagttcgtcagcgccatcaactggcagaaaaccgccacctacgccgacagcgtgaagggccggttcaccatcagccgggacaacgccaagaacagcctgtacctgcagatgaactccctgcgggccgaggacaccgccgtgtactactgcgccgctgtgttccgggtggtggcccccaagacccagtacgactacgattactggggccagggcaccctggtcaccgtgtcatctggcggagggggagaaggcgggggaggggaagggggaggcggcgaagtccagctgctggaatctgggggcggactggtgcagccaggcggctccctcagactgtcttgcgccgcctccggccggatcagcagcatcatccacatggcctggtttagacaggctcccggaaaagaacgcgagctggtgtccgagatctccagagtgggcaccaccgtgtatgccgactccgtgaaaggcagattcacaatctcccgcgacaacagcaagaatactctgtatctccagatgaatagcctgaagcccgaagatacagccgtctactattgcaacgccctgcagtacgagaagcacggcggagccgactattggggacagggaacactcgtgacagtgtctagctgatga